(19) United States
(12) Patent Application Publication
(10) Pub. No.: US 2022/0387173 A1
(43) Pub. Date: Dec. 8, 2022

(54) Expandable Anchor For Prosthetic Mitral Valve

(71) Applicant: Tendyne Holdings, Inc., St. Paul, MN (US)

(72) Inventors: Alec King, Maple Grove, MN (US); Philip Osterbauer, Wyoming, MN (US); Tracee Eidenschink, Wayzata, MN (US); Preston James Huddleston, Maplewood, MN (US); Theodore Paul Dale, Corcoran, MN (US); Amy Marie Danielson, White Bear Lake, MN (US); Son Mai, North Branch, MN (US)

(73) Assignee: Tendyne Holdings, Inc., St. Paul, MN (US)

(21) Appl. No.: 17/752,022

(22) Filed: May 24, 2022

Related U.S. Application Data
(60) Provisional application No. 63/195,770, filed on Jun. 2, 2021.

(51) Int. Cl. A61F 2/24

(52) U.S. Cl. CPC A61F 2/243; A61F 2/2466; A61F 2220/0008

(57) ABSTRACT

A prosthetic heart valve system may include a prosthetic heart valve, a tether having a first end configured to connect to the prosthetic heart valve and a second end, and a collapsible and expandable anchor. The anchor includes a center rim configured to couple to the second end of the tether. The anchor further includes a plurality of struts extending radially outward from the center rim forming an inner dome that is concave toward a heart when the anchor is in a resting configuration implanted on an apex of the heart. The anchor further includes a plurality of wings extending radially outward from the struts.

### CROSS-REFERENCE TO RELATED APPLICATIONS

The present application claims the benefit of the filing date of U.S. Provisional Patent Application No. 63/195,770 filed Jun. 2, 2021, the disclosure of which is hereby incorporated by reference herein in its entirety.

### BACKGROUND

Valvular heart disease, and specifically aortic and mitral valve disease, is a significant health issue in the United States. Annually, approximately 90,000 valve replacements are performed in the United States. Traditional valve replacement surgery, the orthotopic replacement of a heart valve, is an “open heart” surgical procedure. Briefly, the procedure necessitates a surgical opening of the thorax, initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated with the procedure, largely due to the necessity for extra-corporeal circulation. Further, open heart surgery is often poorly tolerated in elderly patients. Thus, if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated with the native mitral valve and thus a greater level of difficulty with regard to inserting and anchoring the replacement prosthesis.

Recent developments in the field have provided devices and methods for mitral valve replacement with reduced invasion and risk to the patient. Such devices typically include a prosthetic valve disposed within the native valve annulus and held in place with an anchor seated against an exterior surface of the heart near the apex, and such anchors must be at least a certain size to seat against the heart with adequate security. Methods of implanting such devices therefore typically require providing an intercostal puncture of significant size to accommodate the anchor. Trauma to the patient increases as a function of the diameter of the puncture. Accordingly, methods and devices for anchoring a prosthetic heart valve that reduce the diameter of any intercostal puncture, or avoid the need for an intercostal puncture altogether, would improve patient outcomes.

### BRIEF SUMMARY

According to an aspect of the disclosure, a prosthetic heart valve system includes a prosthetic heart valve, a tether, and a collapsible and expandable anchor. The tether may have a first end and a second end, and the prosthetic heart valve may be configured to receive the first end of the tether. The anchor may have a center rim configured to couple to the second end of the tether. The anchor may further have a plurality of struts extending radially outward from the center rim forming an inner dome that is concave toward a heart when the anchor is in a resting configuration implanted on an apex of the heart. The anchor may further have a plurality of wings extending radially outward from the struts.

According to another aspect of the disclosure, a prosthetic heart valve system may include a prosthetic heart valve, a tether, and a collapsible and expandable anchor. The tether may have a first end configured to connect to the prosthetic heart valve and a second end. The anchor may have a leading face and a trailing face. The second of the tether may be configured to couple directly to the leading face and may not directly couple to the trailing face of the body, the tether configured to transition the anchor from a resting configuration to a tether configuration by tensioning the tether to apply a tensioning force directly to the leading face.

According to another aspect of the disclosure, a method of implanting an anchor on an external surface of a heart for securing a prosthetic heart valve within the heart may include the steps of collapsing the anchor into a collapsed delivery configuration within a delivery tube, the anchor having a leading face and a trailing face, a tether being attached to the leading face; advancing the delivery tube into a ventricle of the heart; after advancing the delivery tube into the ventricle, passing a distal end of the delivery tube through a ventricular wall of the heart; and after passing the distal end of the delivery tube through the ventricular wall, deploying the anchor from the distal end of the delivery tube onto an apex of the heart by tensioning the tether to restrain movement of the leading face relative to the ventricular wall deploying remaining portions of the anchor to limit a distance which the anchor protrudes beyond the ventricular wall during deployment.

### BRIEF DESCRIPTION OF THE DRAWINGS

FIG. 1 is an exploded view of an exemplary prosthetic cardiovascular valve.

FIG. 2 is an opened and flattened view of an unexpanded inner frame of the prosthetic valve of FIG. 1.

FIGS. 3 and 4 are side and bottom views, respectively, of the inner frame of FIG. 2 in an expanded configuration.

FIG. 5 is an opened and flattened view of an unexpanded outer frame of the prosthetic valve of FIG. 1.

FIGS. 6 and 7 are side and top views, respectively, of the outer frame of FIG. 5 in an expanded configuration.

FIGS. 8-10 are side, front, and top views, respectively, of an assembly of the inner frame of FIGS. 2-4 and the outer frame of FIGS. 5-7, all in an expanded configuration.

FIG. 11A is a perspective view of an anchor for the prosthetic valve of FIG. 1.

FIG. 11B is an axial view of the anchor of FIG. 11.

FIG. 12 is a side view of the anchor for the prosthetic valve of FIG. 1 according to another arrangement.

FIG. 13 is a perspective view of the anchor of FIG. 11 in a partially everted state.

FIG. 14 illustrates a trans-jugular insertion of a delivery tube for the anchor of FIG. 11.

FIG. 15 illustrates a trans-femoral insertion of the delivery tube of FIG. 14.

FIG. 16 illustrates the delivery tube of FIGS. 14 and/or 15 extending through a wall of a heart.

FIGS. 17-20 illustrate the anchor of FIG. 11 in progressive stages of deployment from the delivery tube of FIGS. 14 and 15.

FIGS. 21A and 21B illustrate the delivery tube being retracted from the prosthetic valve of FIG. 1 and the anchor of FIG. 11.

FIG. 22 illustrates the valve of FIG. 1 implanted in a heart.

FIGS. 23A and 23B are perspective views of a frame for the anchor of FIG. 11.

FIG. 24 is an axial view of the anchor of FIG. 11 with the frame of FIGS. 23A and 23B installed.

FIGS. 25A and 25B are perspective views of an anchor according to another embodiment of the disclosure in a resting configuration and a tethered configuration, respectively.

FIG. 26A-C are perspective views of alternate embodiments of the anchor of FIG. 25A.

FIGS. 27A and 27B illustrate the anchor of FIG. 25A in progressive stages of deployment from a delivery tube.

FIG. 28 illustrates an alternate method of deployment of the anchor of FIG. 25A from a delivery tube.

FIG. 29 is a perspective view of the anchor of FIG. 25A in an implanted tethered configuration on the heart.

FIG. 30A is an axial view of an anchor according to another embodiment of the disclosure.

FIG. 30B is an axial view of an anchor according to another embodiment of the disclosure.

FIG. 31A is a perspective view of the anchor of FIG. 30A stretched axially.

FIG. 31B is a schematic view of an anchor according to another embodiment of the disclosure.

FIGS. 32A and 32B are perspective views of the anchor of FIG. 30A including a locking disc and a cinch tether.

FIG. 33A is an X-ray view of the anchor of FIG. 30A in a collapsed configuration

FIGS. 33B-E illustrate the anchor of FIG. 30A in progressive stages of deployment from a delivery tube.

FIG. 34 is a perspective view of the anchor of FIG. 30A in a deployed configuration with a fabric patch.

FIGS. 35A-D are schematic side views of alternate embodiments of the anchor of FIG. 30A having a fabric patch.

FIGS. 35E and 35F are schematic top and side views, respectively, of the anchor of FIG. 30A having a fabric cuff.

FIGS. 35G and 35H are schematic side views of alternate embodiments of the anchor of FIG. 30A having a fabric patch and a fabric cuff.

FIG. 35I is an axial view of the anchor of FIG. 30A, in a partially expanded condition, including piecewise fabric coupled thereto.

FIG. 35J is a perspective view of the anchor of FIG. 35I in the partially collapsed condition.

FIG. 35K is a side view of the anchor of FIG. 35I in the partially collapsed condition.

FIGS. 35L-M are axial and perspective views, respectively, of the anchor of FIG. 35I in an expanded condition.

FIG. 35N is a close-up perspective view of the anchor of FIG. 35I in a radially compressed condition.

FIGS. 35O-P are perspective views of the anchor of FIG. 30A in a partially collapsed condition including piecewise fabric coupled thereto according to another embodiment of the disclosure.

FIG. 36A is a schematic view of the anchor of FIG. 30A including a locking disc and a cinch tether.

FIG. 36B is a schematic view of an alternate embodiment of the anchor of FIG. 36A including a slipknot in the cinch tether.

FIGS. 37A and 37B are schematic views of the anchor of FIG. 32A implanted on a heart in uncinched and cinched configurations, respectively.

FIG. 38A is a perspective view of an anchor according to another embodiment of the disclosure.

FIG. 38B is a perspective view of the anchor of FIG. 38A including a fabric patch.

FIG. 39 is a perspective view of the anchor of FIG. 38A including a fabric patch implanted on a heart.

FIGS. 40A-C are perspective views of varying degrees of axial compression applied to a tube used to form the anchor of FIG. 38A.

FIG. 40D is a schematic close-up view of a hub of an anchor according to another embodiment of the disclosure.

FIG. 40E is a schematic view of the hub of FIG. 40D in a compressed configuration.

FIG. 41A is a schematic view of a block used to form the shape of the anchor of FIG. 38A.

FIGS. 41B and 41C illustrate stages of shaping the anchor of FIG. 38A within the block of FIG. 41A.

FIG. 42A is a schematic view of the anchor of FIG. 38A formed with a fabric patch thereon.

FIGS. 42B and 42C are schematic views of the anchor of FIG. 38A including a stretchable bag in compressed and deployed configurations, respectively.

FIGS. 43A-C illustrate the anchor of FIG. 38A in progressive stages of deployment from a delivery tube.

### DETAILED DESCRIPTION

As used herein, the term “proximal,” when used in connection with a delivery device or components of a delivery device, refers to the end of the device closer to the user of the device when the device is being used as intended. On the other hand, the term “distal,” when used in connection with a delivery device or components of a delivery device, refers to the end of the device farther away from the user when the device is being used as intended. As used herein, the term “leading,” when used in connection with a device, delivery device or components thereof, refers to the end of the device closer to the direction in which the device is being translated. On the other hand, the term “trailing,” when used in connection with a device, delivery device or components thereof, refers to the end of the device farther away from the direction in which the device is being translated. As used herein, the terms “substantially,” “generally,” “approximately,” and “about” are intended to mean that slight deviations from absolute are included within the scope of the term so modified. Unless otherwise stated, like reference numerals refer to like elements throughout the disclosure.

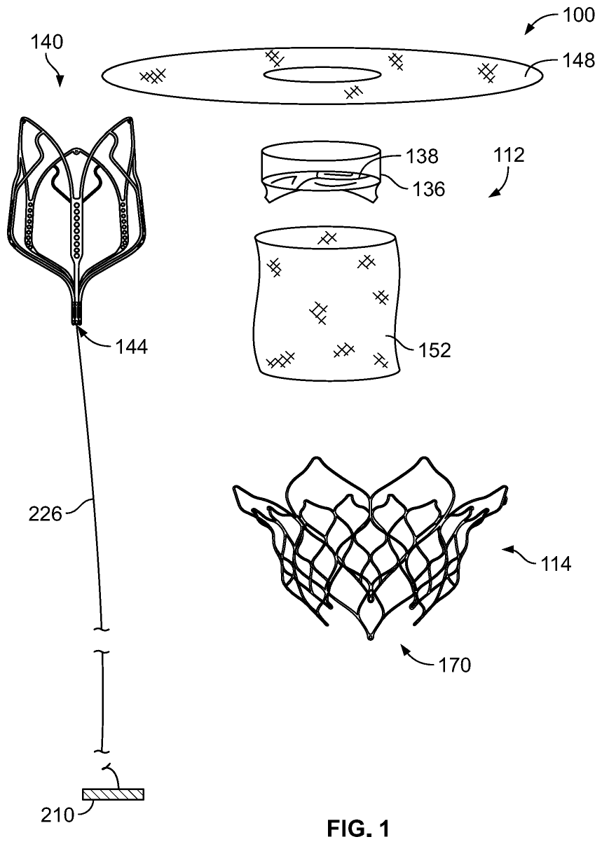

An exemplary prosthetic heart valve 100 as may be used with various embodiments of the present disclosure is shown in an exploded view in FIG. 1. Valve 100 includes an inner structure or assembly 112 and an outer structure or assembly 114. Valve 100 may be coupled to a tether 226 and a collapsible tether anchor 210.

Inner assembly 112 includes an inner frame 140, outer cylindrical wrap 152, and leaflet structure 136 (including articulating leaflets 138 that define a valve function). Leaflet structure 136 may be sewn to inner frame 140, and may use parts of inner frame 140 for this purpose. Inner assembly 112 is disposed and secured within outer assembly 114, as described in more detail below.

Outer assembly 114 includes outer frame 170. Outer frame 170 may also have in various embodiments an outer frame cover of tissue or fabric (not pictured), or may be left without an outer cover to provide exposed wireframe to facilitate in-growth of tissue. Outer frame 170 may also have an articulating collar or cuff (not pictured) covered by a cover 148 of tissue or fabric.

Tether 226 is connected to valve 100 by inner frame 140. Thus, inner frame 140 includes tether connecting or clamping portion 144 by which inner frame 140, and by extension valve 100, is coupled to tether 226.

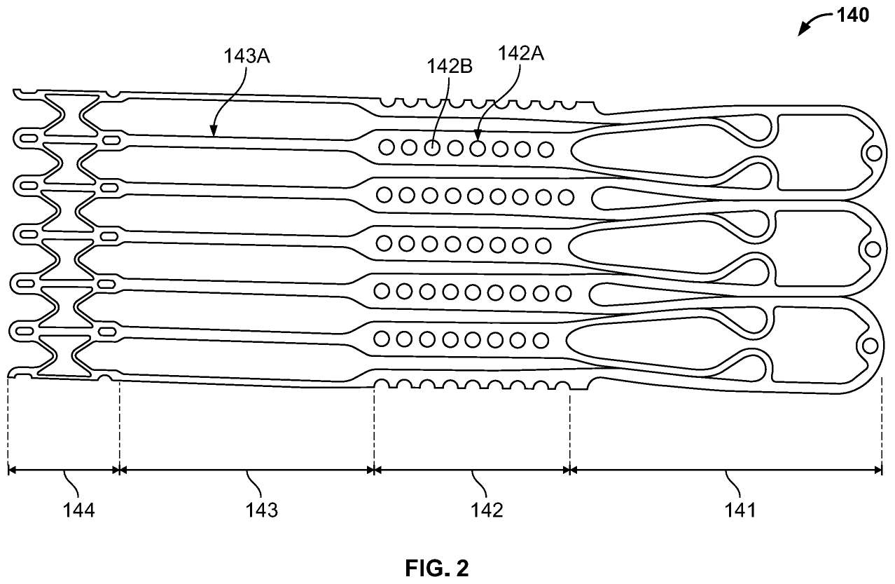

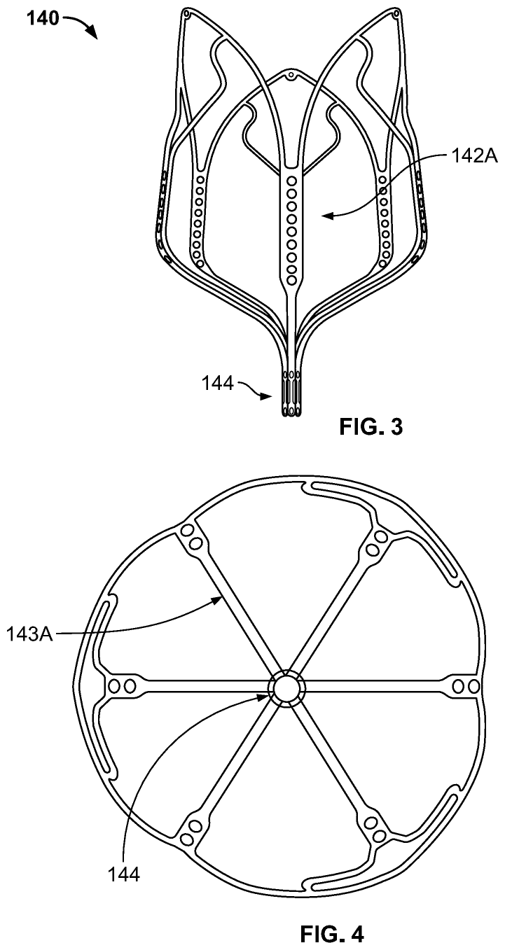

Inner frame 140 is shown in more detail in FIGS. 2-4. Inner frame 140 can be formed from a milled or laser-cut tube of a shape-memory material such as, for example, nitinol. Inner frame 140 is illustrated in FIG. 2 in an undeformed, initial state, i.e., as milled or laser-cut, but cut longitudinally and unrolled into a flat sheet for ease of illustration. Inner frame 140 is shown fully deformed, i.e., to the final, deployed configuration, in the side view and bottom view of FIGS. 3 and 4, respectively. Inner frame 140 can be divided into four portions corresponding to functionally different portions of inner frame 140 in final form: apex portion 141, body portion 142, strut portion 143, and tether connecting portion 144. Strut portion 143 includes six struts, such as strut 143A, which connect body portion 142 to connecting portion 144. A greater or lesser number of struts is contemplated herein.

Connecting portion 144 includes longitudinal extensions of the struts, connected circumferentially to one another by pairs of micro-V's. Connecting portion 144 is configured to be radially collapsed by application of a compressive force, which causes the micro-V's to become more deeply V-shaped, with each pair of vertices moving closer together longitudinally and the open ends of the V shapes moving closer together circumferentially. When collapsed, connecting portion 144 can clamp or grip one end of tether 226, either connecting directly onto a tether line (e.g., braided filament line) or onto an intermediate structure, such as a polymer or metal piece that is, in turn, firmly fixed to the tether line. The foregoing is merely exemplary and other techniques can be used to connect tether 226 to connecting portion 144.

In contrast to connecting portion 144, apex portion 141 and body portion 142 are configured to be expanded radially. Strut portion 143 forms a longitudinal connection, and radial transition, between the expanded body portion 142 and the compressed connecting portion 144.

Body portion 142 includes six longitudinal posts, such as post 142A, although the body portion may include a greater or lesser number of such posts. The posts can be used to attach leaflet structure 136 to inner frame 140, and/or can be used to attach inner assembly 112 to outer assembly 114, such as by connecting inner frame 140 to outer frame 170. In the illustrated example, posts 142A include apertures 142B through which connecting members (such as suture filaments and/or wires) can be passed to couple the posts to other structures.

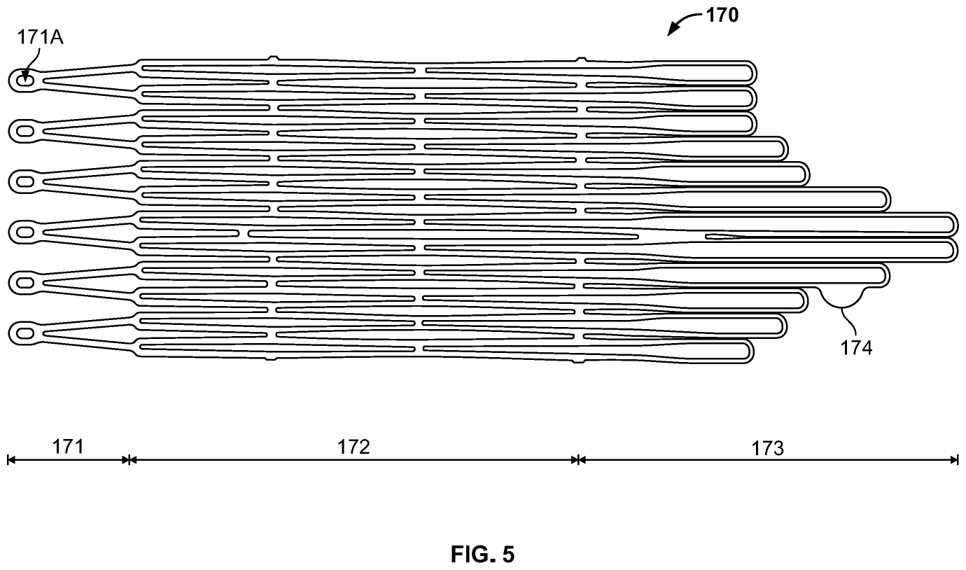

Outer frame 170 of valve 100 is shown in more detail in FIGS. 5-7. Outer frame 170 can be formed from a milled or laser-cut tube of a shape-memory material such as, for example, nitinol. Outer frame 170 is illustrated in FIG. 5 in an undeformed, initial state, i.e., as milled or laser-cut, but cut longitudinally and unrolled into a flat sheet for ease of illustration. Outer frame 170 can be divided into a coupling portion 171, a body portion 172, and a flared portion 173, as shown in FIG. 5. Coupling portion 171 includes multiple openings or apertures 171A by which outer frame 170 can be coupled to inner frame 140, as discussed in more detail below.

Flared portion 173 may include an indicator 174. In one example, indicator 174 is simply a broader portion of the wire frame element of flared portion 173, i.e., indicator 174 is more apparent in radiographic or other imaging modalities than the surrounding wireframe elements of flared portion 173. In other examples, indicator 174 can be any distinguishable feature (e.g., protrusion, notch, etc.) and/or indicia (e.g., lines, markings, tic marks, etc.) that enhance the visibility of the part of flared portion 173 on which it is formed, or to which it is attached. Indicator 174 can facilitate the implantation of the prosthetic valve by providing a reference point or landmark that the operator can use to orient and/or position the valve (or any portion of the valve) with respect to the native valve annulus or other heart structure. For example, during implantation, an operator can identify (e.g., using echocardiography) indicator 174 when the valve 100 is situated in a patient's heart. The operator can therefore determine the location and/or orientation of the valve and make adjustments accordingly.

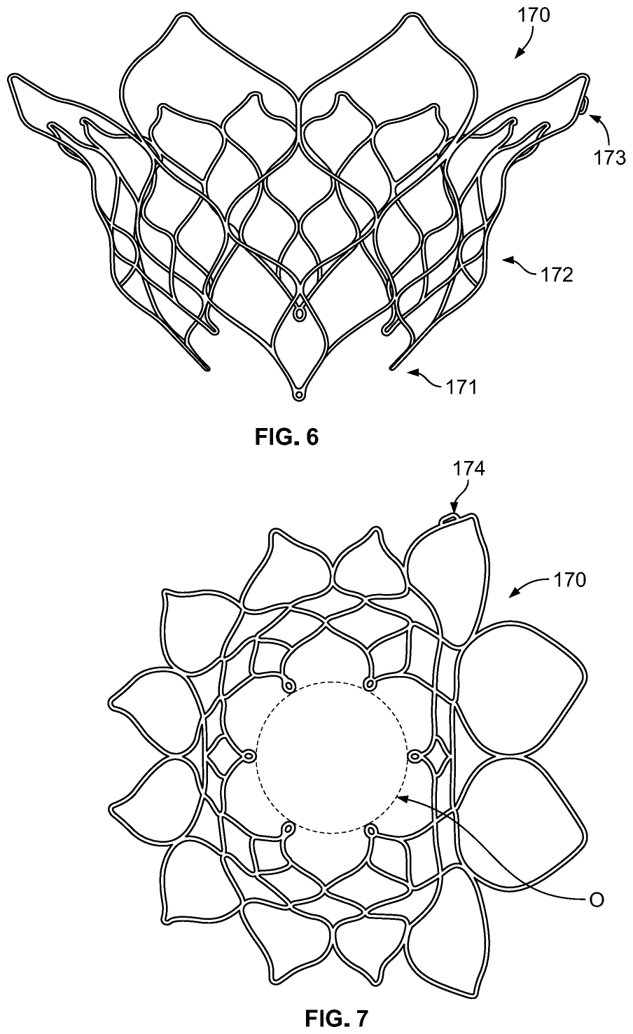

Outer frame 170 is shown fully deformed, i.e., to the final, deployed configuration, in the side view and top view of FIGS. 6 and 7, respectively. As best seen in FIG. 7, the lower end of coupling portion 171 forms a roughly circular opening (identified by “O” in FIG. 7). The diameter of this opening preferably corresponds approximately to the fully deformed diameter of body portion 142 of inner frame 140 to facilitate the coupling together of these two components of valve 100.

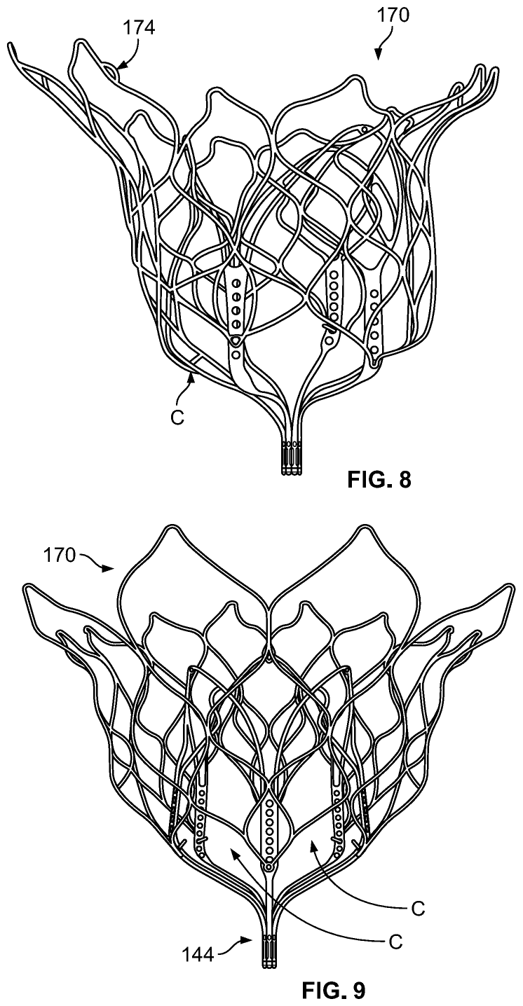

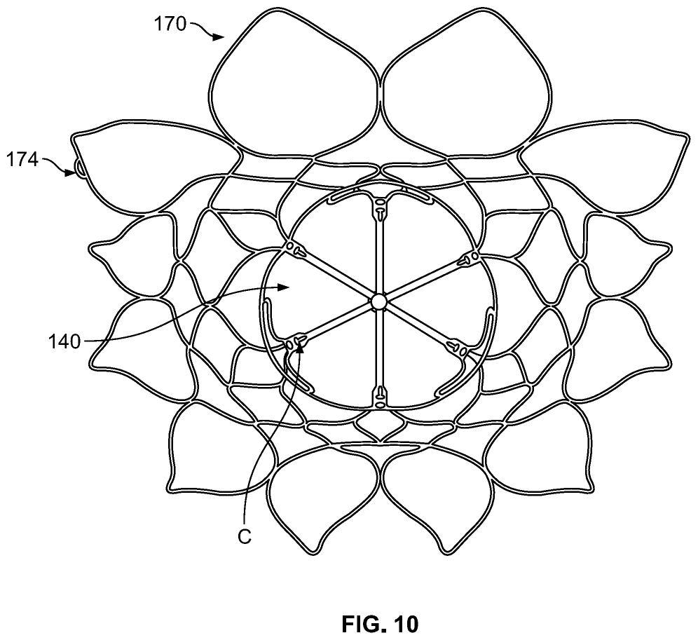

Outer frame 170 and inner frame 140 are shown coupled together in FIGS. 8-10 in front, side, and top views, respectively. The two frames collectively form a structural support for a valve leaflet structure, such as leaflet structure 136 in FIG. 1. The frames support leaflet structure 136 in the desired relationship to the native valve annulus, support the coverings for the two frames to provide a barrier to blood leakage between the atrium and ventricle, and couple to the tether 226 (by the inner frame 140) to aid in holding the prosthetic valve in place in the native valve annulus by the connection of the free end of the tether and tether anchor 210 to the ventricular wall, as described more fully below. The two frames are connected at six coupling points (representative points are identified as “C”). In this embodiment, the coupling of the frames is implemented with a mechanical fastener, such as a short length of wire, passed through an aperture 171A in coupling portion 171 of outer frame 170 and a corresponding aperture 142B in a longitudinal post 142A in body portion 142 of inner frame 140. Inner frame 140 is thus disposed within the outer frame 170 and securely coupled to it.

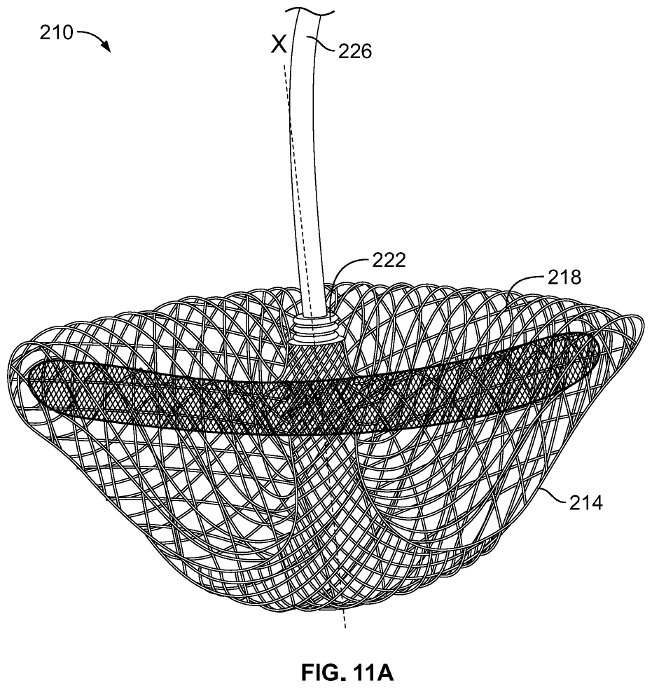

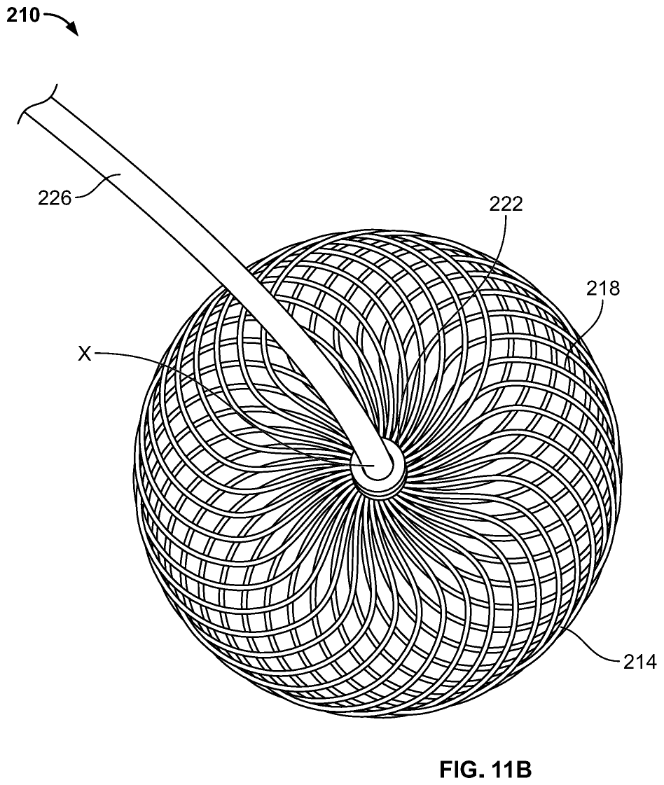

An exemplary anchor 210 for a prosthetic mitral heart valve is illustrated in FIGS. 11A and 11B. Anchor 210 includes a first disc 214 and a second disc 218, both provided by a wire mesh and centered on an axis X. First disc 214 is offset from second disc 218 in a first direction along axis X. First disc 214 and second disc 218 are each biased toward a dome-shaped resting configuration that is concave toward a second direction along axis X, the second direction being opposite the first direction. The resting configuration of first disc 214 extends far enough in the second direction along axis X to partially overlap second disc 218.

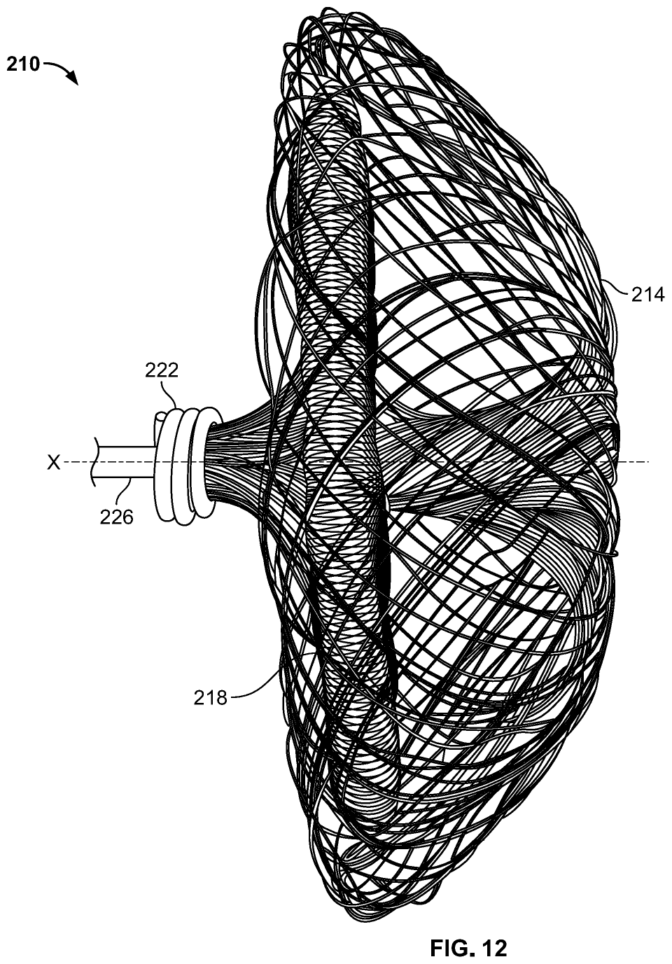

It should be understood that the illustrated dome shapes are merely exemplary, and first disc 214 and second disc 218 may be biased differently. For example, either or both of first disc 214 and second disc 218 may be biased toward a resting configuration that is convex toward the second direction or generally planar. Further, the first disc 214 and second disc 218 may be biased to different resting configurations. In one example, the first disc 214 may be biased toward a dome-shaped resting configuration that is concave toward the second direction while the second disc 218 is biased toward a generally planar configuration having about the same diameter location as the widest part of the dome-shaped resting configuration of the first disk 214, as shown in FIG. 12. In further examples, the first disc 214 may be concave toward the second direction while the second disc 218 is concave toward the first direction such that the concave portions of the first and second disc face each other. In the arrangement shown in FIG. 12, second disc 218 is generally planar in shape with a shallow concavity toward the first direction near the center of second disc 218.

Anchor 210 also includes a cuff, anchor cap 222, or other connector for holding the braids of the anchor together and/or for gripping a tether 226, which may be connected to a prosthetic heart valve. It is also contemplated that tether 226 may extend through anchor cap 222 and couple and/or anchor to a distal portion of the braids. Anchor cap 222 is offset from second disc 218 in the second direction along axis X. One-way gripping features, such as angled teeth, within anchor cap 222 may permit anchor 210 to slide along tether 226 in the second direction, but not the first direction.

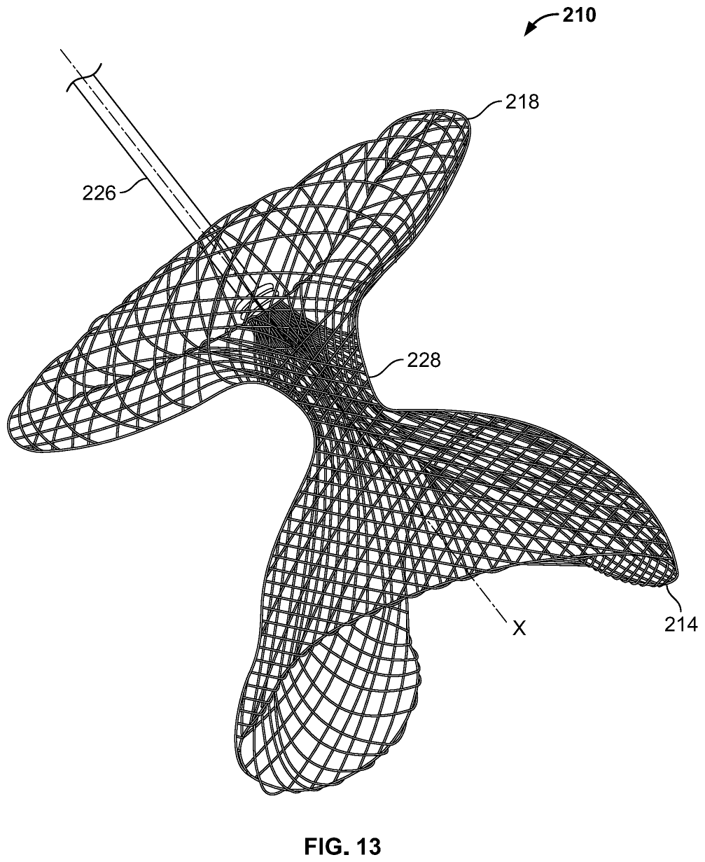

Anchor 210 is flexible, as illustrated in FIG. 13, which shows anchor 210 with the first disc 214 everted from its resting configuration. First disc 214 is connected to second disc 218 by a neck 228 extending between first disc 214 and second disc 218. In the illustrated example, neck 228 is centered on axis X, but in other examples neck 228 may be radially offset from axis X. First disc 214, second disc 218, and neck 228 may all be constructed from a single continuous piece or tube of wire mesh, e.g., nitinol braid. The wire mesh may be formed from a plurality of strands or wires braided into various three-dimensional shapes and/or geometries to engage tissues, or from one or more sheets cut to provide mesh, such as by laser. In one example, the wires form a braided metal fabric that is resilient, collapsible and capable of heat treatment to substantially set a desired shape. One class of materials which meets these qualifications is shape-memory alloys, such as nitinol. The wires may comprise various materials other than nitinol that have elastic and/or memory properties, such as spring stainless steel, trade named alloys such as Elgiloy® and Hastelloy®, CoCrNi alloys (e.g., tradename Phynox®), MP35N®, CoCrMo alloys, or a mixture of metal and polymer fibers. Depending on the individual material selected, the strand diameter, number of strands, and pitch may be altered to achieve the desired shape and properties of anchor 210. Shape memory materials such as nitinol may be particularly suitable for anchor 210 in that shape memory material construction enables anchor 210 to consistently return to an intended shape after being compressed and deployed. In other arrangements, anchor 210 may be covered by or may incorporate other flexible biocompatible material, such as a fabric.

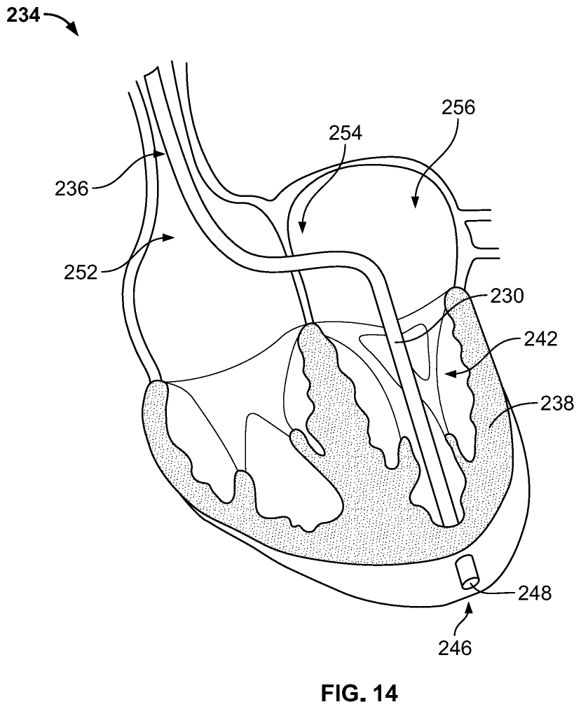

FIG. 14 shows a trans-jugular insertion of an at least partially flexible delivery tube 230 for anchor 210 and valve 100. Delivery tube 230 may be formed of any known material for building catheters, including biocompatible metals such as steel, and may be part of a steerable or flexible catheter system. Delivery tube 230 may include an inflexible portion near its distal end to facilitate the intended puncture of tissue and guidance of valve 100. Delivery tube 230 is inserted through the patient's jugular vein (not shown), then through superior vena cava 236, right atrium 252, atrial septum 254, left atrium 256, native mitral valve 260, and into left ventricle 242. Tube 230 exits left ventricle 242 through ventricular wall 238 at or near the apex 246 of heart 234. A retractable puncturing device (not shown) and a retractable atraumatic tip (not shown) may extend from the distal open end 248 of tube 230 in alternate stages of insertion of tube 230. The puncturing device may produce openings through atrial septum 254 and ventricular wall 238 while the atraumatic tip may act to prevent injury to other tissue. Once delivery tube 230 has been fully inserted, the distal open end 248 of tube 230 is positioned outside of ventricular wall 238. The trans-jugular insertion of tube 230 may be accomplished by any of variety of methods, such as, for example, guiding tube 230 along a guide wire, such as a shape-memory guide wire, inserted through the jugular vein. The flexible nature of anchor 210 allows trans-jugular delivery of anchor 210 through tube 230. Because tube 230, anchor 210, and valve 100 all reach heart 234 from the jugular vein, valve 100 and anchor 210 may be delivered and implanted without any intercostal puncture. Although various collapsible and expandable anchors are described for use with a prosthetic mitral valve being delivered transseptally, the anchors could also be used in transapical deliveries, with modifications to the connections between the tether, prosthetic heart valve, and anchor as may be required.

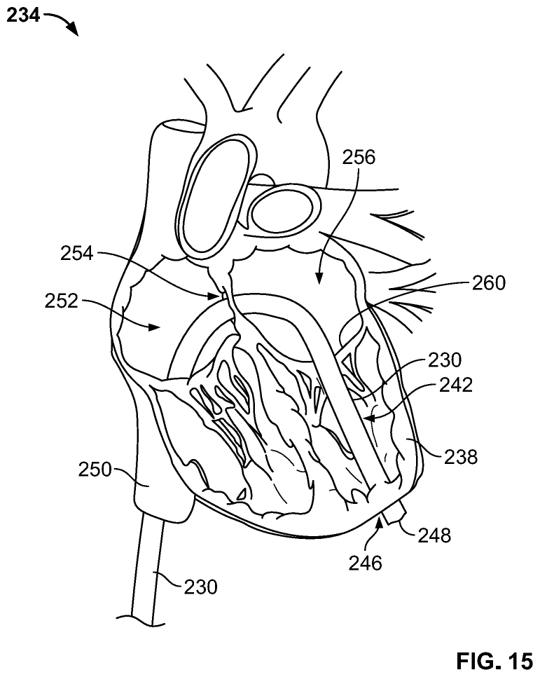

FIG. 15 shows a trans-femoral insertion of tube 230. Tube 230 enters heart 234 through inferior vena cava 250, travels through right atrium 252, and punctures septum 254 to enter left atrium 256. Tube 230 is advanced from left atrium 256 through native mitral valve 260, left ventricle 242, and ventricular wall 238 such that the open end 248 of the tube is positioned outside of wall 238 at or near apex 246. As with trans-jugular insertion, guidance of tube 230 during trans-femoral insertion may be accomplished using a variety of methods, including guidance along a guide wire.

The trans-jugular and trans-femoral insertions described above are merely exemplary. It should be understood that tube 230 could be guided toward heart 234 using any suitable method known in the art.

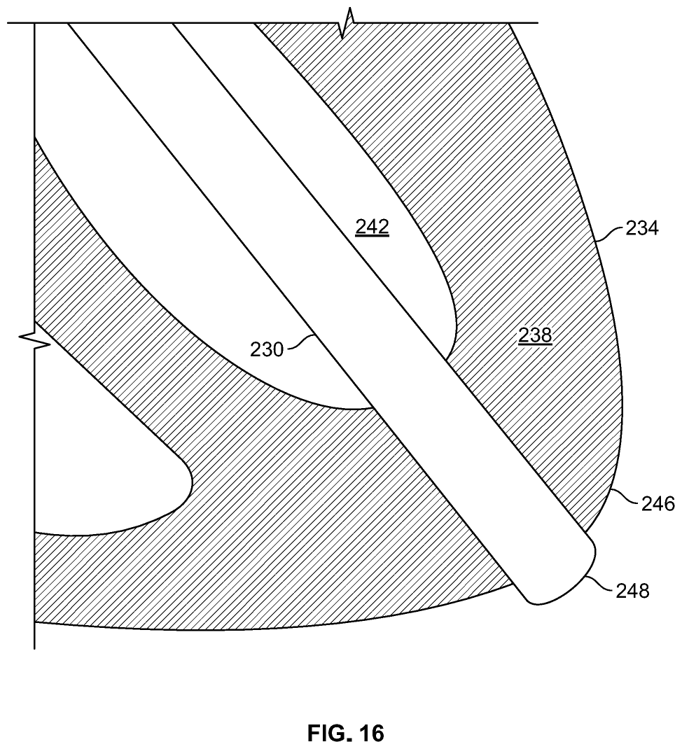

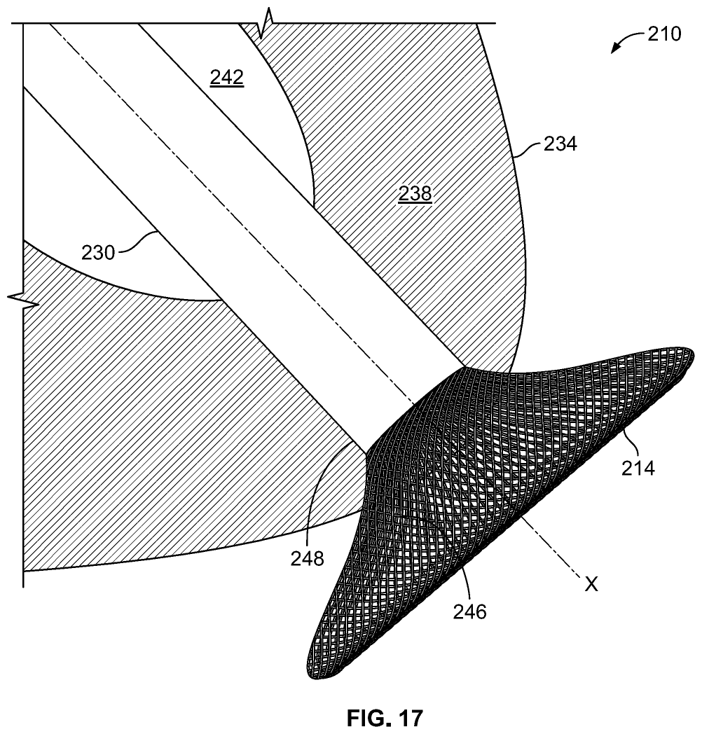

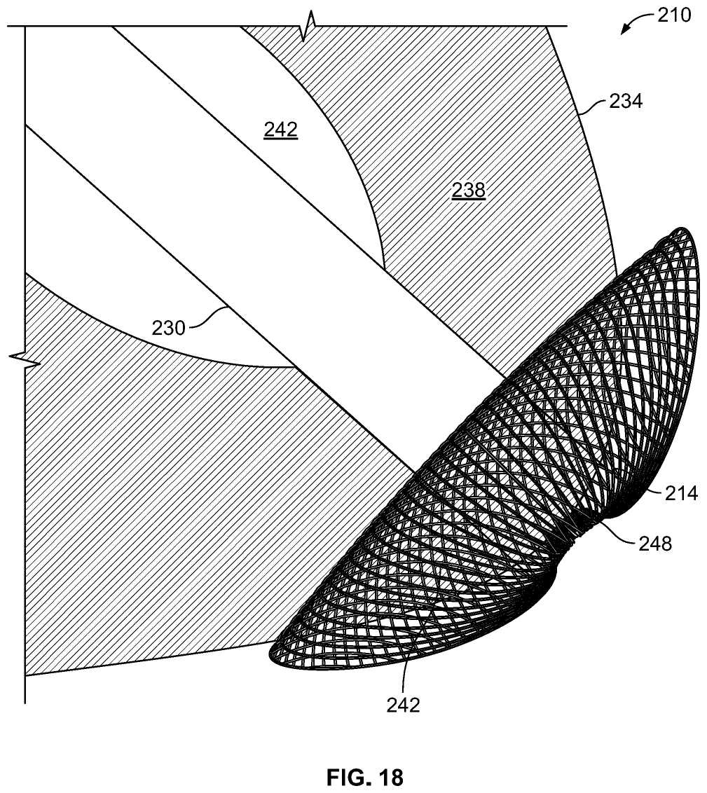

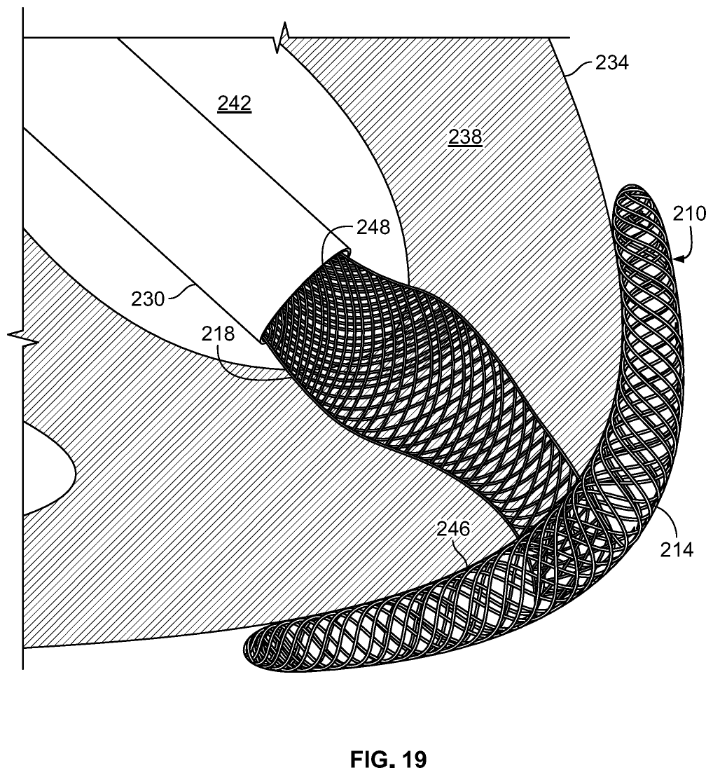

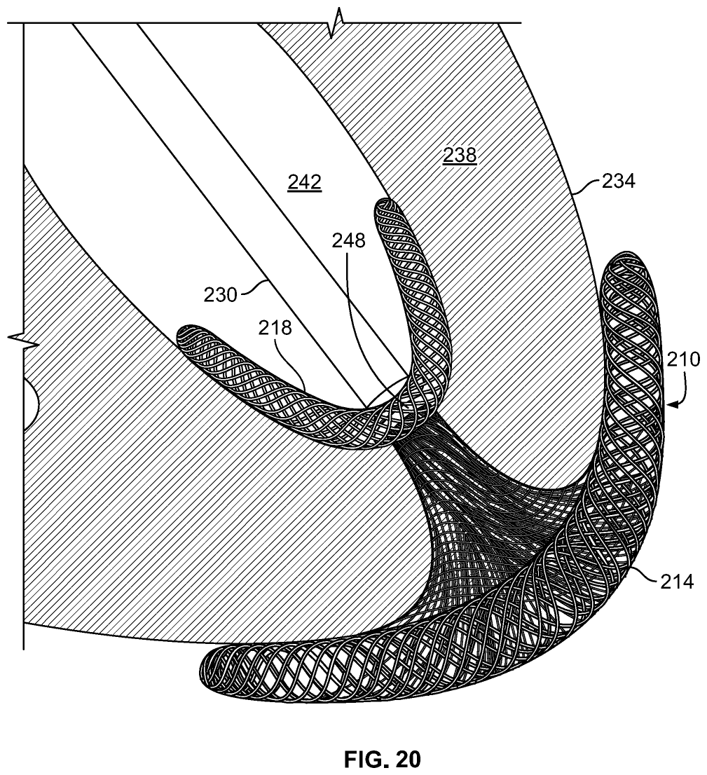

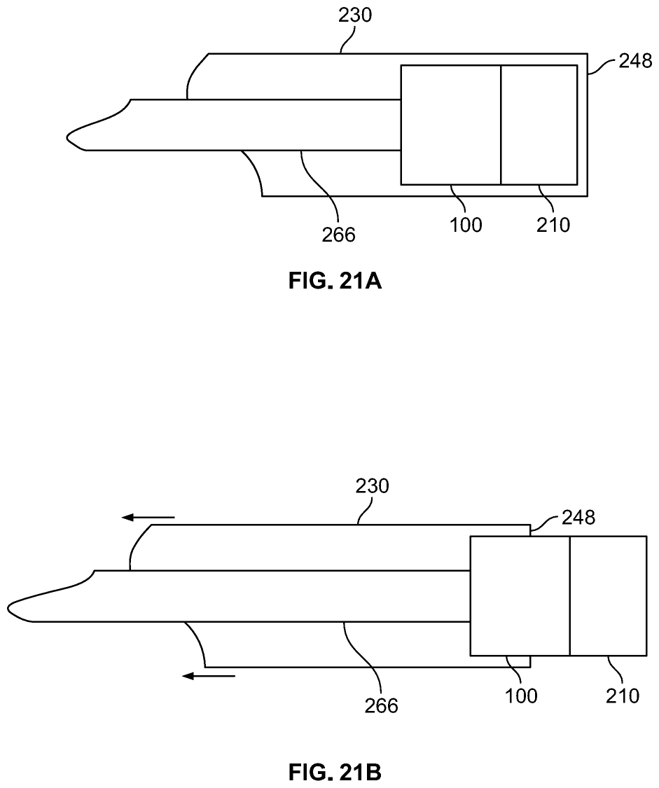

FIGS. 16-20 illustrate anchor 210 in progressive stages of deployment from the open end 248 of tube 230. Tube 230 is shown in a distalmost position in FIG. 16, with open end 248 positioned outside of heart 234. Tube 230 may be retracted while anchor 210 is forced to remain in place, such as by a reversal of a typical Bowden cable arrangement. For example, a semi-rigid cable or wire 266 may be inserted through tube 230 to contact the proximal end of valve 100, as shown in FIG. 21A. Pulling tube 230 proximally relative to wire 266 causes valve 100 and anchor 210 to deploy out from the open end 248 of tube 230, as shown in 21B. As shown in FIG. 17, retracting tube 230 while preventing anchor 210 from retreating with the tube into heart 234 causes first disc 214 of anchor 210 to deploy out from the open end 248 of tube 230 and expand radially relative to axis X. Upon further retraction of tube 230 or advancement of the anchor 210 (or a combination of both movements), the bias of first disc 214 causes it to curve back onto the outer apex 246 of heart 234, as shown in FIG. 18. Further retraction of tube 230 in FIG. 19 allows second disc 218 to deploy and expand radially relative to axis X within left ventricle 242 until second disc 218 opens to press against an inner side of wall 238, as shown in FIG. 20. Pressure against wall 238 results from the elastic bias of first disc 214 and second disc 218 toward certain resting positions as described above with regard to FIGS. 11A, 11B, and 12. First disc 214 and second disc 218 pressing on opposite sides of wall 238 causes anchor 210 to grip wall 238. Such progressive expansion from within a narrow tube results in anchor 210 adequately securing valve 100 to ventricular wall 238 without requiring an intercostal puncture through the patient's chest.

According to alternative embodiments or arrangements, tube 230 may be retracted while anchor 210 is held in place by a cord (not shown) connected to anchor 210 and extending out from an intercostal incision in the patient's chest. In an embodiment employing this method, tube 230 may extend into left ventricle 242 but not entirely or at all through ventricular wall 238, and anchor 210 may be deployed by pulling anchor 210 out of tube 230 and through wall 238 using the cord.

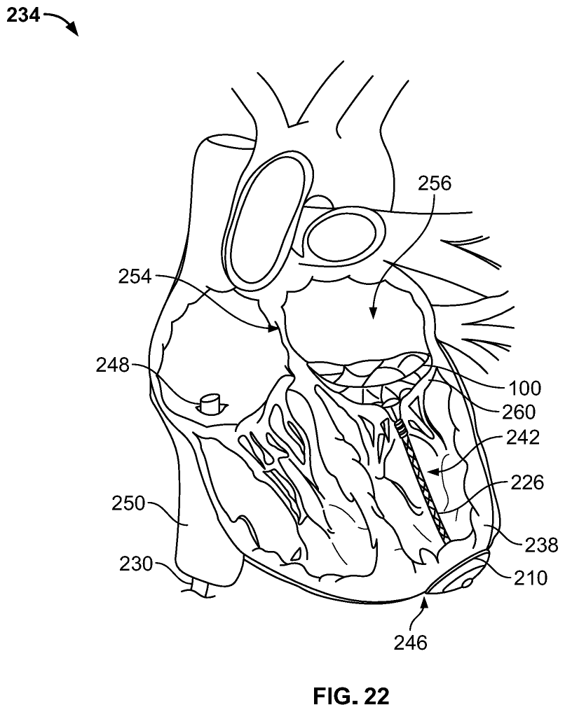

FIG. 22 illustrates valve 100 implanted in heart 234 with anchor 210 seated at or near the apex 246 of heart 234. Tube 230 has been withdrawn from heart 234, through inferior vena cava 250 in the illustrated example, leaving valve 100 behind.

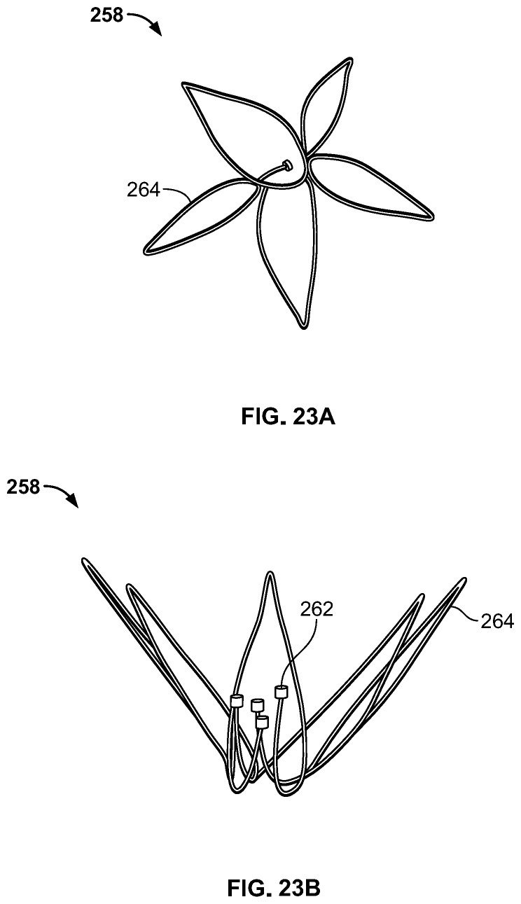

FIGS. 23A and 23B illustrate a flexible reinforcing frame 258 for anchor 210. Frame 258 optionally may be installed as illustrated to reinforce anchor 210. Frame 258 is constructed from a laser cut sheet or wire that is both thicker and less flexible than the wire mesh of anchor 210 (or simply less flexible), but frame 258 may be constructed from any suitable elastically deformable biocompatible material, such as nitinol. Frame 258 tends toward a resting configuration in which leaves 264 of frame 258 are arranged to form a cone shape. Frame 258 may be inverted from the cone shape, but will return to the cone shape upon release from external forces. It is also contemplated that frame 258 may be used as the anchor by itself. For example, tether 226 may pass through frame 258 and couple to a distal portion of frame 258 in a manner similar to the embodiments described below in greater detail.

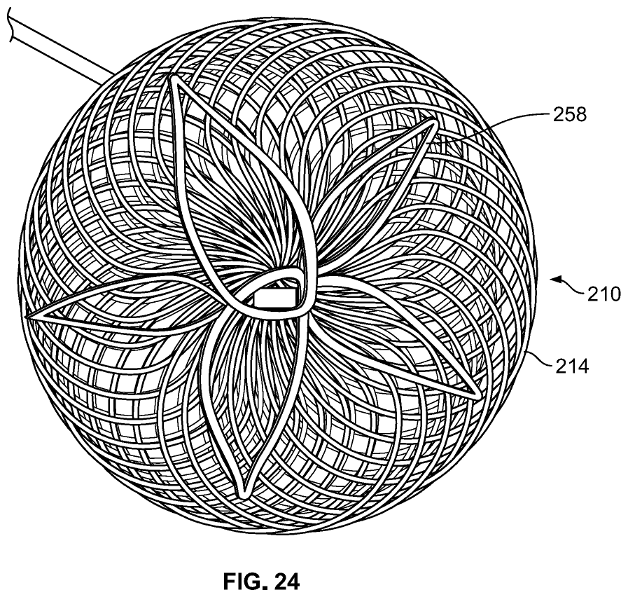

FIG. 24 shows frame 258 installed on anchor 210. Frame 258 is disposed over first disc 214, on a side of first disc 214 opposite second disc 218. Tabs 262 extend from frame 258 axially through the neck 228 of anchor 210 and are bound to tether 226 by anchor cap 222. In such an arrangement, frame 258 would be inverted and compressed radially inward so as to extend in the first direction relative to anchor 210 when anchor 210 is compressed within tube 230. For example, first disc 214 and frame 258 may be inverted to extend in the first direction along axis X within tube 230 prior to delivery. Frame 258 will fold back toward the second direction and into the cone shape after release from tube 230. Frame 258 further includes tabs 262 that, in the rest configuration of frame 258, extend into an interior of the cone shape from a point at which leaves 264 intersect. As anchor 210 is deployed, frame 258 expands radially outward and folds backward toward the second direction and ventricular wall 238 to push first disc 214 onto wall 238. Tabs 262 may extend into anchor cap 222, and frame 258 may be connected to anchor 210 by anchor cap 222 gripping tabs 262.

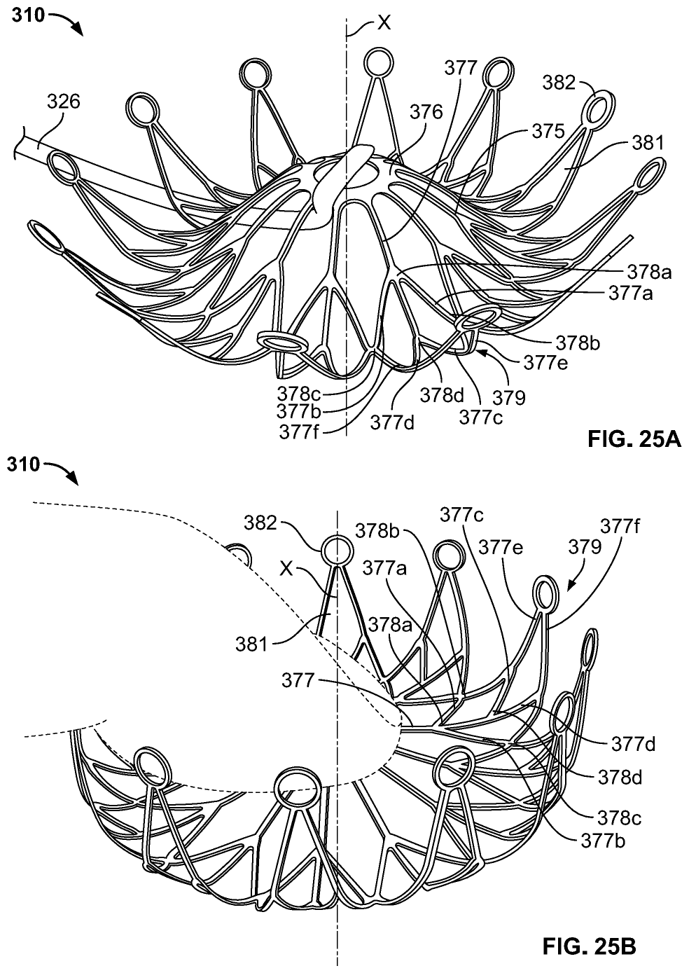

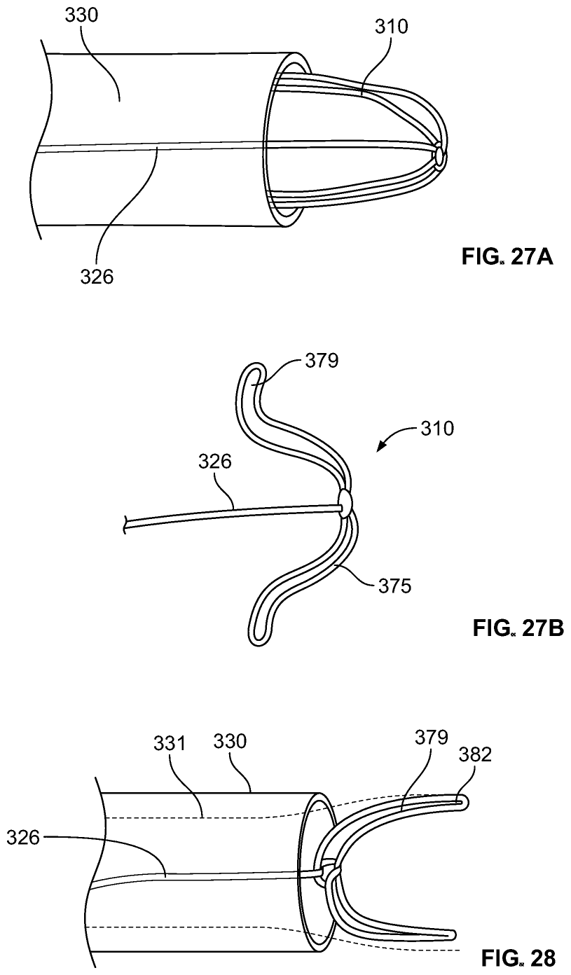

FIGS. 25A-B illustrate an anchor 310 according to an alternative embodiment of the disclosure. Anchor 310 is configured to be delivered to the apex of the heart in a manner similar to the above-described delivery of anchor 210. That is, anchor 310 may be compressed within a delivery tube (as will be described with reference to FIGS. 27A-B) to be delivered transseptally, e.g., through trans-jugular or trans-femoral insertions as described above with reference to FIGS. 14-15. Anchor 310 may have a compressed delivery configuration while disposed within the delivery tube, a resting expended configuration when released or deployed from the delivery tube, and a tensioned configuration when tension is applied to tether 326. After anchor 310 is deployed and delivered, the anchor may be coupled to a prosthetic heart valve (such as valve 100) via tether 326 by, e.g., positioning the valve in the native valve annulus and affixing the valve to tether 326 with a suitable tension between the valve and anchor 310.

Anchor 310 is shown in FIG. 25A in a resting expanded configuration. Anchor 310 may form an inner dome 375 centered on an axis X and concave in a first direction. The first direction may be toward the heart relative to anchor 310 when anchor 310 is in an implanted configuration, and may correspond to the bottom of the view in FIG. 25A. Inner dome 375 includes center rim 376 centered on and extending circumferentially around axis X. Radial struts 377 extend radially outward and in the first direction from center rim 376. Each radial strut 377 includes a first transverse strut 377a and a second transverse strut 377b extending from a common intersection point 378a located along radial strut 377 in directions away from each other to form a “V” shape. The “V” shape is formed at each radial strut 377 such that a transverse strut extending from each radial strut intersects with a transverse strut extending from an adjacent radial strut, defining lateral intersection points, e.g., 378b, 378c. Wings 379 extend from struts 377, 377a, 377b in the radially outward direction and in a second direction opposite the first direction, each wing defining a free end, or a radially terminal end, of the anchor 310. Wings 379 are formed by third transverse strut 377c, fourth transverse strut 377d, first wing strut 377e and second wing strut 377f. Third and fourth transverse struts 377c, 377d extend from a common intersection point 378d located at a radial end of radial strut 377 in directions away from each other to form a “V” shape. First wing strut 377e extends from first lateral intersection point 378b, and second wing strut 377f extends from second lateral intersection point 378c. Third transverse strut 377c intersects first wing strut 377e at a point along first wing strut 377e. Fourth transverse strut 377d intersects second wing strut 377f at a point along second wing strut 377f. Third transverse strut 377c, fourth transverse strut 377d, first wing strut 377e and second wing strut 377f form diamond-shaped cell 381. Wings 379 further include eyelets 382 extending from a junction point of wing struts 377e, 377f such that eyelets 382 are positioned radially outward of the struts. The shape of anchor 310 in the resting configuration (e.g., the portion between inner dome 375 and wings 379) creates atraumatic contact points where struts contact the heart. Applying tension to inner dome 375 as shown may cause the inner dome 375 to begin to flatten an amount, creating a disc of contact with the myocardium and distribute the pressure applied to the heart to the region surrounding the puncture formed to deploy anchor 310. That is, whereas a flat anchor subject to tension may apply pressure onto the punctured hole, the illustrated anchor may minimize pressure applied to the puncture and thereby minimize necrosis of the heart. In addition, the combination of (i) the position of the wings 379 pointing away from the heart and (ii) the inner dome 375 being spaced from the heart tissue prior to tension being applied may reduce the likelihood that the anchor 310 is pulled through the apical puncture upon application of tension. In comparison, an anchor that has a generally similar shape to anchor 310, but is instead flat prior to being tensioned, may be more likely to be pulled through the apical puncture upon tensioning.

Anchor 310 is configured to couple to tether 326 at center rim 376, and tether 326 may extend from anchor 310 in the first direction. In some examples, tether 326 may be tied to anchor 310 by passing through center rim 376 from the first direction and forming a knot to prevent retraction as shown in FIG. 25A, although any coupling mechanism may be appropriate. Anchor 310 is shown in FIG. 25B in a tethered configuration. It should be understood that typically anchor 310 would assume the tethered configuration via tension from tether 326 in the first direction, however for illustration purposes, a finger is shown applying a similar force to inner dome 375 to create the image of anchor 310 in the tethered configuration. In the tethered configuration, the concavity of inner dome 375 may be reduced or eliminated and struts 377 may extend radially outward in a direction substantially perpendicular to the first and second directions (e.g. substantially perpendicular to axis X). Further, in the tethered configuration, wings 379 may deflect such that wings 379 pivot further in the second direction and less in the outward direction. The degree of change in shape of anchor 310 is directly affected by the amount of tension applied to tether 326. That is, the greater the force of tension applied, the farther center rim 376 is pulled in the first direction and the greater the pivot action of wings 379.

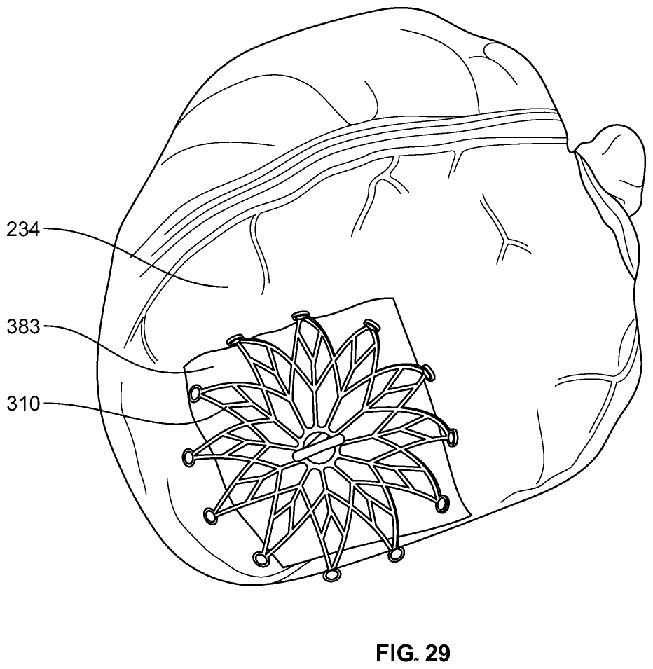

As shown in FIG. 29, anchor 310 may further include a fabric 383 sutured (or otherwise coupled) thereto to substantially abut the myocardium and provide a more complete seal of the transapical puncture to reduce and/or prevent pericardial effusion. Fabric 383 may be composed of polyurethane terephthalate (PET), nylon, polyurethane, collagen, poly[L-lactide-co-6-caprolactone] (PLLC), combinations thereof, or the like. The contour of fabric 383 may be optimized such that tethered anchor 310 may apply a force to the fabric in the first direction where anchor 310 contacts fabric 383. The force may be distributed uniformly around fabric 383, forming a disc of contact with the myocardium, which may flatten the fabric to prevent dimpling and minimize the distance anchor 310 extends away from the heart.

Anchor 310 may be formed from a milled or laser-cut tubing or flat sheet of a shape-memory material such as, for example, nitinol. Anchor 310 may be resilient, collapsible and capable of heat treatment to substantially set a desired shape. In some examples, anchor 310 is heat set into a contoured profile which may resemble a hemisphere or a bundt pan. Although anchor 310 is shown with a structure being formed from a laser-cut tube of nitinol, in other embodiments, anchor 310 could be constructed from braided wire mesh, similar to that described in connection with anchor 210. In other arrangements, anchor 310 may be covered by or may incorporate other flexible biocompatible material, such as a fabric 383 as shown in FIG. 29 to further seal the heart. It is contemplated that the dimensions of anchor 310 may be adjusted to optimize the mechanical properties. For example, wall thickness, width of struts 377, laser cut pattern (e.g., modifying the shapes of diamond-shaped cells 381), heat-set profile and heat-set parameter may be modified. It is further contemplated that the size, shape and design of anchor 310 may be customized according the size or shape of a heart of a specific patient. The flexibility provided by laser-cutting enables refinement of the anchor design to optimize for delivery profile, force distribution and pull-through resistance.

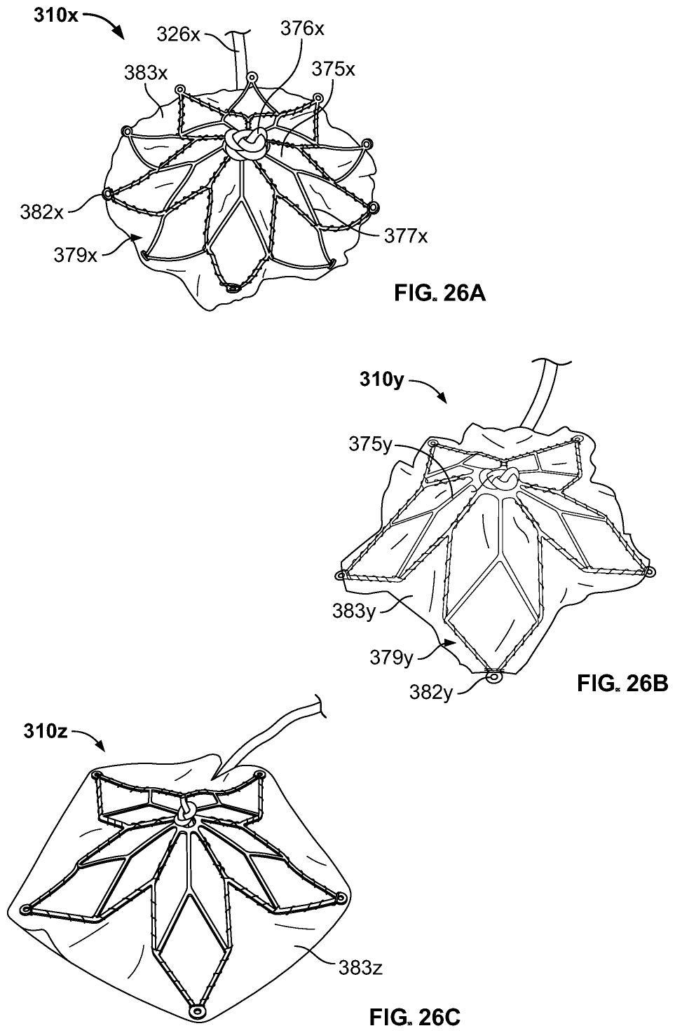

FIG. 26A illustrates an anchor 310x according to another embodiment of the disclosure. Anchor 310x is substantially similar to anchor 310. Anchor 310x includes inner dome 375x, struts 377x and wings 379x forming eyelets 382x. In the resting expanded configuration shown in FIG. 26A, wings 379x extend substantially in the radially outward direction (i.e., perpendicular to the first and second directions). Wings 379x extend slightly in the second direction, substantially less so than wings 379 of anchor 310. Anchor 310x includes a fabric 383x and a tether 326x coupled (e.g., sewn) flatly against fabric 383x and coupled to center rim 376x. FIG. 26B illustrates an anchor 310y according to another embodiment of the disclosure. Anchor 310y is substantially similar to anchor 310x, including inner dome 375y concave in the first direction and wings 379y extending radially outward. Wings 379y extend from inner dome 375y substantially perpendicular to the first and second directions, forming substantially flat contact points at the outer portion of wings 379y where eyelets 382y are formed. Anchor 310y further includes fabric 383y coupled to and covering the proximal face of anchor 310y (i.e., closer to the heart when in the implanted configuration) and a plastic film coupled to and covering the distal face of anchor 310y (i.e., farther from the heart when in the implanted configuration) for sealing. FIG. 26C illustrates an anchor 310z according to another embodiment of the disclosure. Anchor 310z is substantially similar to anchor 310y. Anchor 310z includes fabric 383z coupled to and covering the proximal face of anchor 310z, and a plastic film coupled to and covering the proximal side of fabric 383z (i.e., the fabric is configured to abut the heart in the implanted configuration) for sealing. The plastic film in the above-described embodiments may be formed of, for example, polyurethane (e.g., Thoralon), silicone, polytetrafluoroethylene (PTFE), polyvinyl chloride (PVC), PET, etc.

Anchor 310 may be deployed from delivery tube 330 in a manner substantially similar to anchor 210 described above. Anchor 310 may be collapsed and loaded into delivery tube 330 in the direction of its intended curvature when placed on the heart. In other words, the concavity of inner dome 375 may be oriented in the first direction (i.e., proximally) toward a proximal end of delivery tube 330. Delivery tube 330 (or another separate delivery system apparatus) may puncture the myocardium and anchor 310 may be deployed via a push rod onto the exterior of the heart while coupled to tether 326. As anchor 310 is deployed from tube 330, wings 379 may extend radially outward and anchor 310 may immediately assume the deployed configuration, minimizing the distance into the pleural space required for full deployment. In other examples, strings 331 may be coupled to wings 379 at eyelets 382 as shown in FIG. 28 with strings 331 extending proximally from eyelets 382 to be accessible to a user. Anchor 310 may be inverted, collapsed and loaded into delivery tube 330. In other words, inner dome 375 may be flipped such that inner dome 375 is concave in the second direction (i.e., distal direction). Anchor 310 may be deployed via a push rod, and as eyelets 382 exit delivery tube 330, tension may be applied to strings 331 to hold eyelets 382 in place relative to delivery tube 330. The push rod may be advanced farther to revert inner dome 375 and deploy anchor 310 over the apex of the heart. Anchor 310 is shown in an implanted tethered configuration in FIG. 29. Another option for delivery may include compressing the anchor 310 so that the concavity of the inner dome 375 is oriented in the first direction, as shown in FIG. 27A, but the free ends of the wings 379 point distally. In this configuration, portions of the wings 379 may radially overlap portions of the inner dome 375, potentially leading to a larger profile in the collapsed condition compared to those shown in FIGS. 27A and 28.

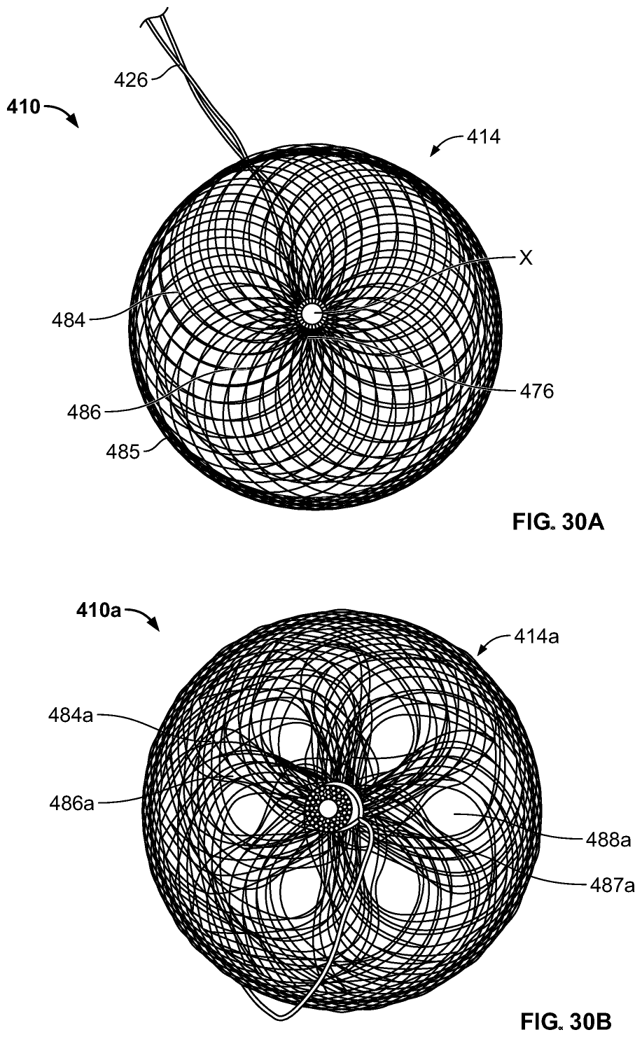

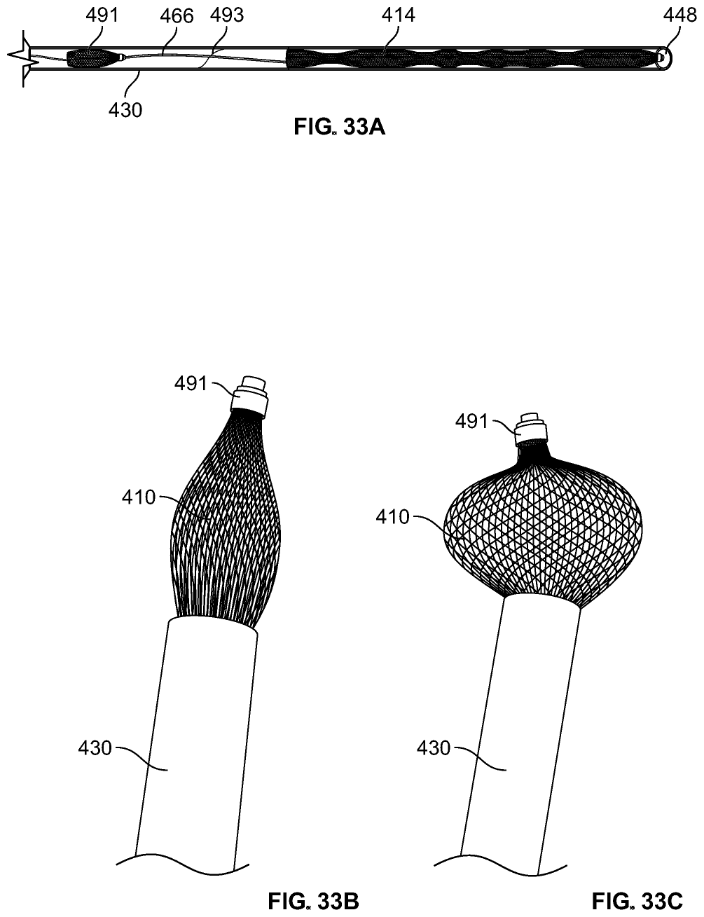

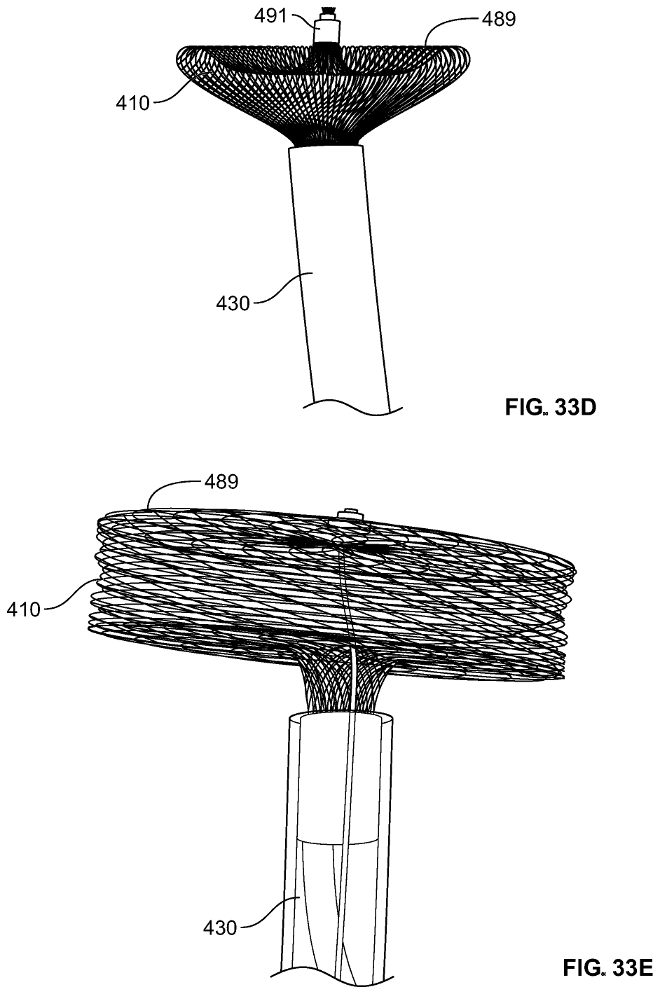

FIG. 30A illustrates an anchor 410 according to another embodiment of the disclosure. Anchor 410 is configured to be delivered to the apex of the heart in a manner similar to the above-described delivery of anchors 210 and 310. That is, anchor 410 may be compressed to be disposed within a delivery tube (as will be described with reference to FIGS. 33A-E) to be delivered transseptally, e.g., through trans-jugular or trans-femoral insertions as described above with reference to FIGS. 14-15. Anchor 410 may have a compressed delivery configuration while disposed within the delivery tube, a resting expanded configuration when released from the delivery tube, and a tensioned configuration when a tension is applied to tether 426.

Anchor 410 is shown in FIG. 30A in a resting expanded configuration. Anchor 410 includes a first disc 414 which may be formed of the same or similar material and means as described above with reference to anchor 210. For example, first disc 414 may be formed of one or more strands of shape-memory wire 484 (e.g., nitinol) which may be braided together to form a mesh, and shaped (e.g., heat set) into a disc-shape centered on axis X. The concentration of the strands of wire 484 may vary in different parts of first disc 414. For example, the strands of wire 484 may be more highly concentrated closer to the perimeter of first disc 414 forming a rim 485, and closer to the radial center of first disc 414 where an inner portion 476 is formed. A middle portion 486 of wire 484 extending radially outward from inner portion 476 and radially inward from rim 485 may be less concentrated with strands of wire 484 relative to inner portion 476 and rim 485. In some examples, as shown in FIG. 30A, the strands of wire 484 may be generally evenly distributed throughout middle portion 486. In other examples, such as first disc 414a shown in FIG. 30B, the strands of wire 484a in middle portion 486a may be distributed such that middle portion 486a forms bridge portions 487a in which the strands of wire 484a are more highly concentrated, thereby also forming openings or pockets 488a in which strands of wire 484a may be less concentrated or absent in the resting expanded configuration, thus forming a wheel-shaped first disc 414a. In other words, the wheel-shaped first disc 414a may include spokes in the form of bridge portions 487a, with gaps between adjacent bridge portions. The concentration of strands of wire 484a forms bundles of wires or bridge portions 487a which may provide increased strength to first disc 414a compared to evenly spaced wires. That is, bridge portions 487a may improve both radial and axial strength of first disc 414a compared to an anchor such as anchor 414 having evenly distributed strands of wire 484. Increased radial strength provides greater resistance to forces applied radially inward, and increased axial strength provides greater resistance to forces applied in the direction of axis X. In the example shown, first disc 414a includes six bridge portions. In some examples, first disc 414a may include between four and ten bridge portions 487a, although any number of bridge portions 487a may be suitable, including zero (or one continuous) bridge portions such as shown in FIG. 30A.

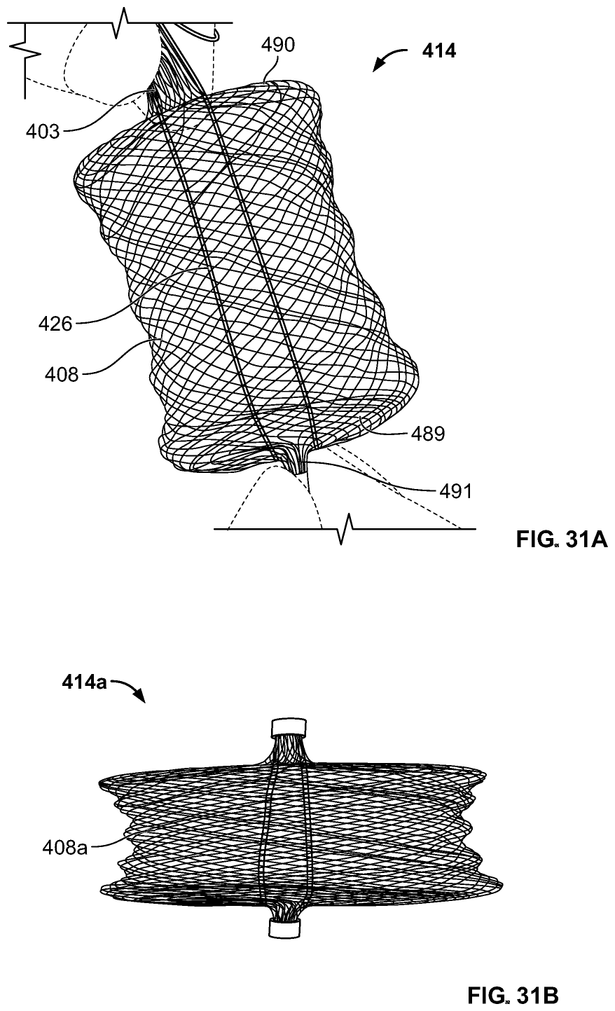

First disc 414 is shown in FIG. 31A in an axially expanded configuration for illustration purposes. First disc 414 defines a leading face 489 disposed and facing away from the heart when anchor 410 is in the implanted configuration. First disc 414 further defines a trailing face 490 disposed and facing toward the heart when anchor 410 is in the implanted configuration. Leading face 489 may include a leading hub 491 disposed within inner portion 476 extending along axis X. First disc 414 may further include a trailing hub 403 on trailing face 490 disposed within center portion 476 extending along axis X. The hubs 491, 403 may help secure gathered free ends of the individual strands braided together to form first disc 414. First disc 414 may further include pleats 408 formed along the radial edge of first disc 414. In some examples, as shown in FIG. 31A, pleats 408 may be formed radially such that the perimeter of first disc 414 is corrugated in the axial direction. In further examples, as shown in FIG. 31B, pleats 408a may be formed axially such that the perimeter of first disc 414a is corrugated in the circumferential direction. Pleats may provide additional structural support to the anchor by causing the braid to stack upon itself, increasing the amount of material along the outer edge of the first disc. Increased material may lead to increased strength around the edge of the first disc, thus leading to an improved ability to disperse force along the edge of the first disc. Increased strength may reduce or prevent dimpling in the center of the first disc. Further, pleats may improve the adjustability of the anchor size and shape by allowing for ease of compression and expansion of the braid. Tether 426 may be coupled (e.g. directly coupled) to leading hub 491 at leading face 489. Tether 426 may merely pass through but not couple (e.g. not directly couple) to trailing hub 403 such that the force from tension on tether 426 is not directly applied to trailing hub 403. In the illustrated embodiment, tether 426 is one string looped around leading hub 491 in two string portions. In other examples, the tether may include one string portion coupled (e.g., tied) to the leading hub. In some examples, the first disc may include between 36 and 144 strands of wire. In further examples, the strands of the wire may have a diameter between 0.005 inches and 0.011 inches.

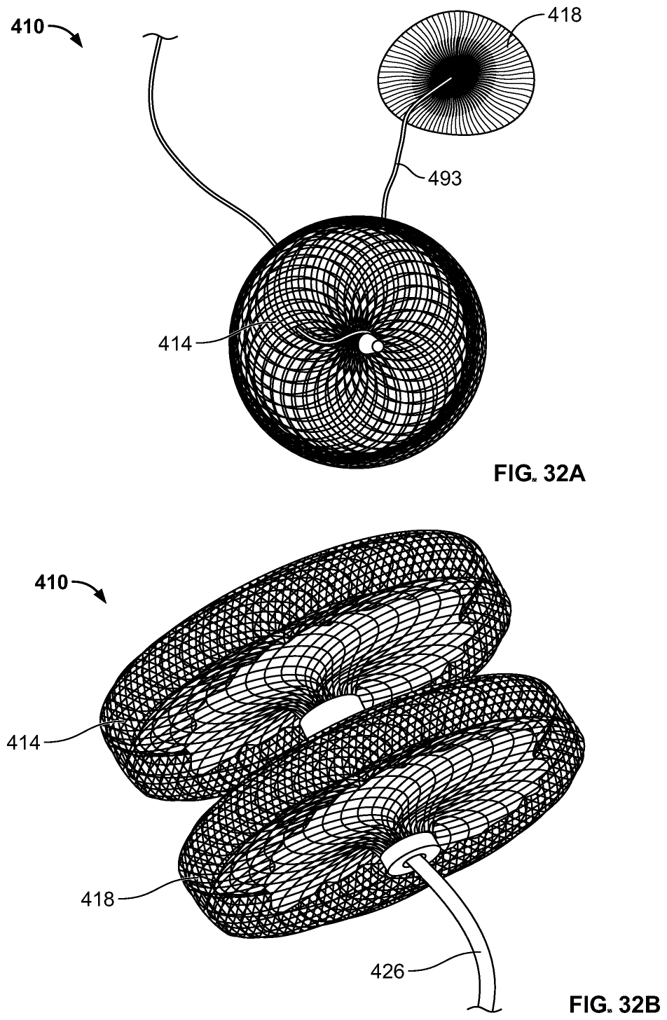

Anchor 410 may include a locking disc 418 similar to second disc 218 described above, although it should be understood that first disc 414 and locking disc 418 may be fully separate structures, and that locking disc 418 is optional. Locking disc 418 may be movably coupled to first disc 414 by cinch tether 493 as shown in FIG. 32A. Locking disc 418 is offset from first disc 414 in the first direction. In other words, locking disc 418 trails first disc 414 and is positioned proximal to first disc 414 in the implanted configuration. Cinch tether 493 may be tensioned by a user to reduce the distance between first disc 414 and locking disc 418, as will be described below in greater detail. Locking disc 418 may be formed of any of the materials described above with reference to first disc 414, such as braided strands of a shape memory wire (e.g., nitinol). First disc 414 and locking disc 418 may alternatively form an integrated single-piece anchor in which the position of the discs are generally fixed relative to each other, as shown in FIG. 32B. The orientations and angles of first disc 414 and locking disc 418 may adjust relative to each other to surround and contour each respective side of ventricular wall 238. Locking disc 418 may be substantially similar in size to first disc 414. In other examples, locking disc 418 may be smaller in size than first disc 414 such as, for example, smaller in radius and wire thickness.

The deployment of anchor 410 from a delivery tube 430 is shown in FIGS. 33A-E. In FIG. 33A, anchor 410 is shown in an X-ray image disposed inside delivery tube 430. Anchor 410 may be deployed from tube 430 by push rod 466. In the illustrated embodiment, anchor 410 includes first disc 414, locking disc 418 and cinch tether 493. Upon being delivered to the left ventricle of the heart, delivery tube 430 (or another component of the delivery system) may puncture the ventricular wall and pass through to deploy first disc 414 outside the heart. In some examples, leading hub 491 of leading face 489 may be sufficiently sharp in the collapsed configuration to puncture the hole in the ventricular wall. Distal open end 448 of delivery tube 430 may be advanced outside the heart into the intercostal space and first disc 414 may be partially deployed from tube 430 as shown in FIG. 33B by translating push rod 466 toward distal open end 448 of tube 430 (or by pulling the tube 430 proximally relative to the push rod 466). As first disc 414 is deployed from the tube 430, tension may be applied to tether 426 as shown in FIG. 33C. The tension on tether 426 may retract or hold substantially in place the leading end of first disc 414 relative to the ventricular puncture as push rod 466 continues to deploy anchor 410 from tube 430. FIG. 33D illustrates first disc 414 substantially deployed from tube 430 with continued tension applied to tether 426, forming leading face 489. In FIG. 33E, first disc 414 is fully deployed from tube 430 in the resting expanded configuration. In examples including a locking disc, the delivery tube may be further retracted relative to the push rod to deploy the locking disc on the inside of the heart wall within the left ventricle to be secured to the heart wall, as described below in greater detail. Delivery tube 430 may then be retracted and tether 426 tensioned so that first disc 414 is secured over the left ventricular apex of the heart. Such a deployment method may minimize the distance beyond the wall of the heart into the intercostal space through which anchor 410 extends during deployment. In other words, by maintaining tension on the tether 426 to restrict the amount of distance the leading face 489 of the anchor 410 may extend beyond the outer surface of the heart, the total distance which the anchor 410 extends beyond the outer surface of the heart during deployment is minimized.

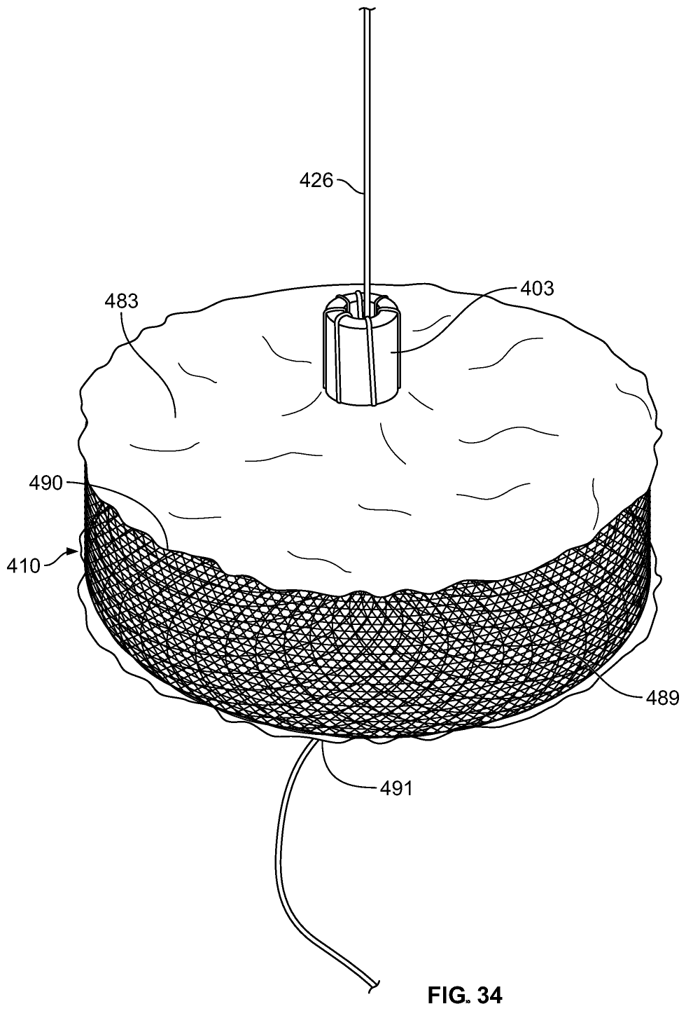

Anchor 410 is shown in FIG. 34 fully deployed from the delivery sheath in an implanted tethered configuration, although it should be understood that the anchor 410 in FIG. 34 is illustrated outside of the heart for purposes of clarity. As described above, tether 426 is coupled to leading hub 491 at leading face 489 and passes through trailing hub 403 at trailing face 490. Applying tension to the leading hub may distribute the force of tension around the entirety of the first disc. The leading face may deflect toward the heart, and such a configuration of the first disc may minimize the force directly on the punctured hole in the heart and distribute the force for improved stability. This distribution of force may also reduce the likelihood that the anchor 410 can pull backwards through the apical puncture, as well as spread the force from tensioning the anchor 410 over a larger surface area of the heart, reducing likelihood of negatively impacting the heart tissue (e.g. via necrosis).

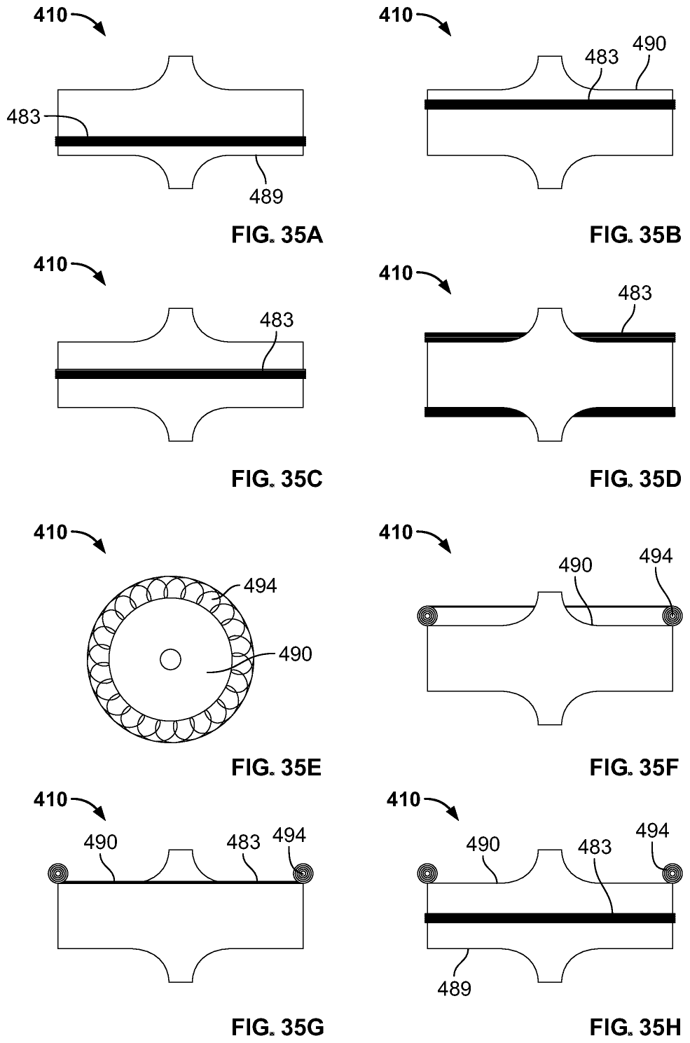

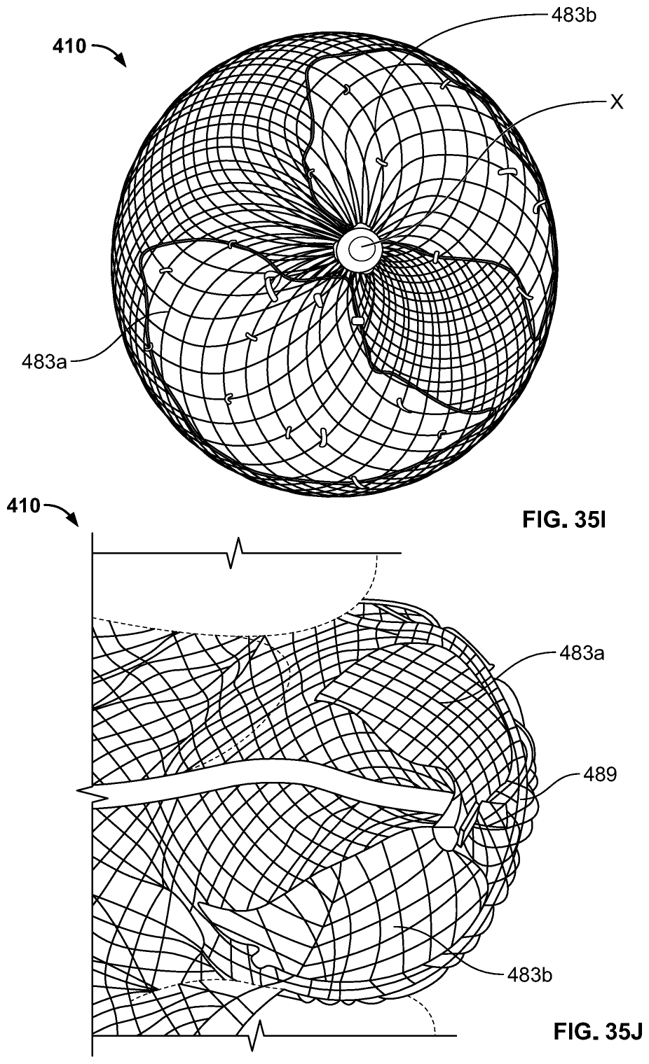

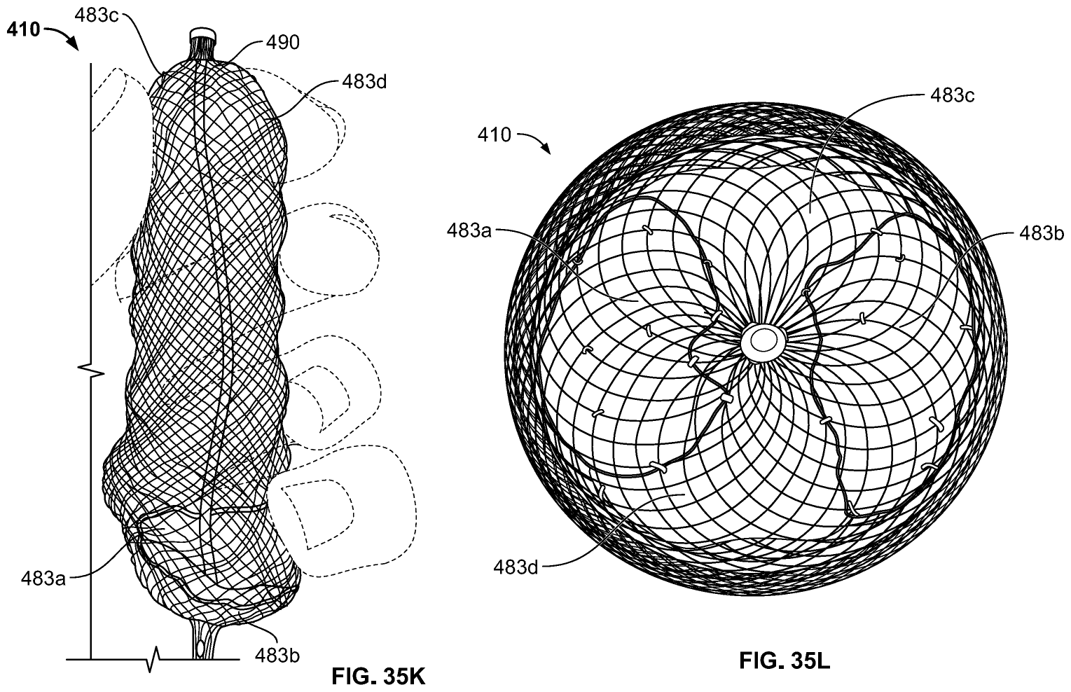

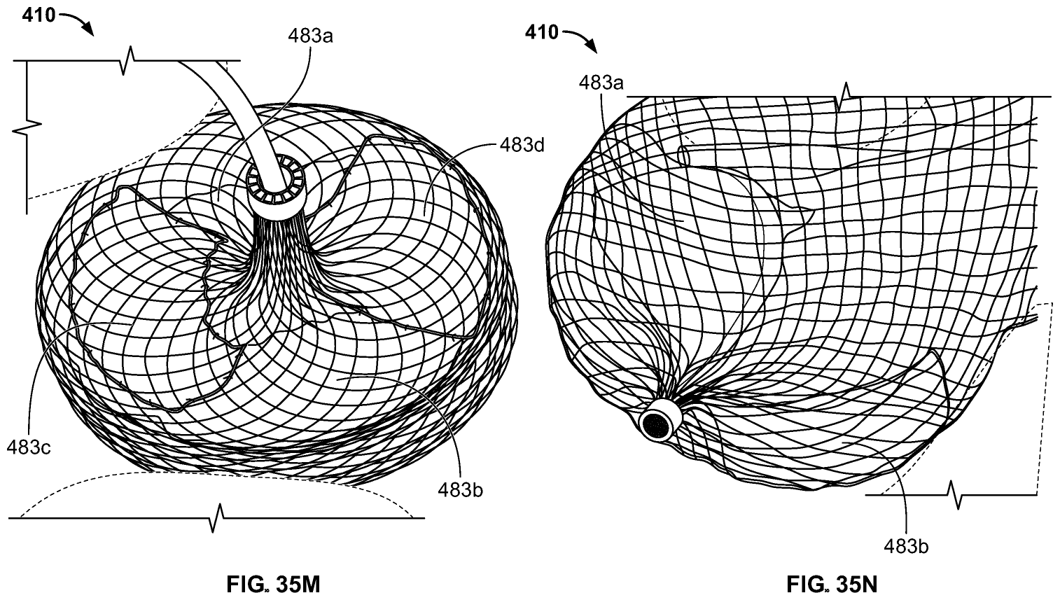

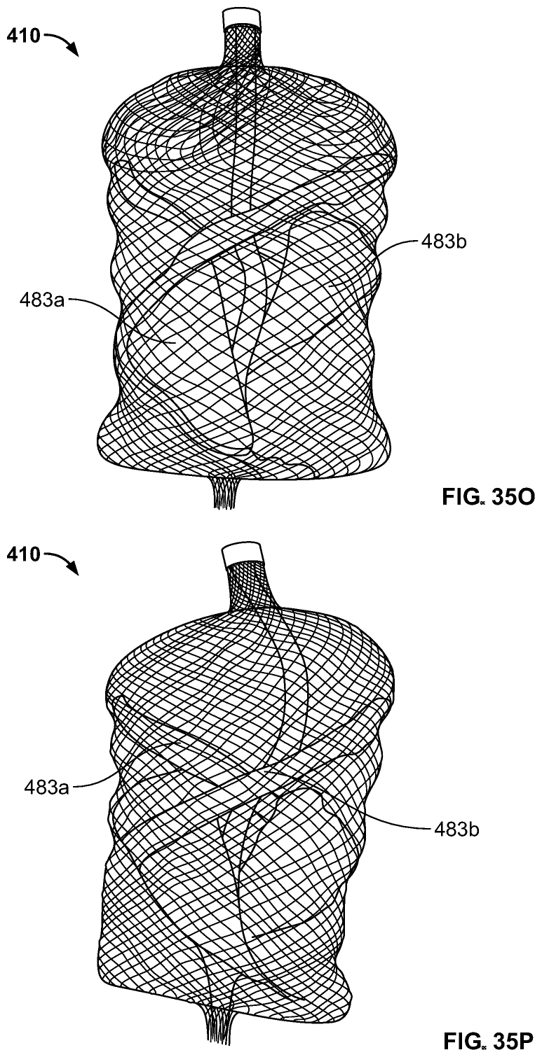

In some examples, anchor 410 may include a fabric patch 483 adjacent trailing face 490 of first disc 414 as shown in FIG. 34. Patch 483 may be disposed between trailing face 490 of first disc 414 and the heart when anchor 410 is in the implanted tethered configuration. Patch 483 may cover a full (or substantially full) cross-sectional area of anchor 410 and may aid in preventing leakage from the heart after the ventricle is punctured for delivery, as well as helping speed tissue ingrowth to seal the puncture. Although patch 483 is illustrated in FIG. 34 on the exterior of first disc 414 adjacent the trailing face 490, patch 483 may be positioned in various alternate or additional locations. For example, patch 483 may be positioned internally within the first disc 414 proximate to leading face 489 of first disc 414 as shown in FIG. 35A, proximate to trailing face 490 of first disc 414 as shown in FIG. 35B, or substantially centered between the leading and trailing faces as shown in FIG. 35C. Alternatively, patch 483 may be positioned externally on anchor 410 on trailing face 490 of first disc 414, leading face 489 of first disc 414, or both as shown in FIG. 35D. It should be understood that the above embodiments may be combined in any desired fashion, for example including one or more internally positioned patches with one or more externally positioned patches.

In some examples, anchor 410 may include a cuff 494. Cuff 494 may be an annular fabric extending partially or completely around the perimeter of first disc 414, e.g., disposed on trailing face 490 as shown in FIG. 35E-F. In other examples, the cuff may be disposed on the leading face. It is also contemplated that anchor 410 may include a combination of cuff 494 and patch 483. For example, in FIG. 35G, patch 483 is positioned internally within first disc 414 proximate to trailing face 490 and cuff 494 is disposed on trailing face 490 external to first disc 414. With reference to FIG. 35H, patch 483 is positioned internally within first disc 414 substantially in the middle of trailing face 490 and leading face 489, and cuff 494 is disposed on trailing face 490 external to first disc 414. In further examples, the anchor may include a patch and cuff both disposed externally on the first disc. It should be understood that anchor 410 may include a cuff 494 and/or patch 483 in accordance with any of the embodiments described herein applied to either or both of first disc 414 and locking disc 418.

In further examples, anchor 410 may include a piecewise patch arrangement. FIGS. 35I-N illustrate various views of anchor 410 according to one example of the disclosure while transitioning between the collapsed condition and the expanded condition, the anchor shown in various stages of the transition and having patch pieces coupled thereto. Anchor 410 is shown in FIG. 35I in a partially expanded condition in which leading face 489 is expanded but trailing face 490 is still collapsed, the anchor 410 having at least first patch piece 483a and second patch piece 483b. Each patch piece (e.g., 483a, 483b) may be coupled to (e.g., stitched around the perimeter of the patch piece) and positioned within anchor 410 to cover a portion of the cross-sectional area of the anchor. In some examples, each patch piece may be oriented within anchor 410 such that the patch piece extends generally along a plane perpendicular to longitudinal axis X. In such examples, each patch piece may be positioned at a different axial location such that some or none of the patch pieces are aligned along the same axial cross-section of the anchor taken perpendicular to longitudinal axis X. The specific embodiment shown in FIG. 35I includes two patch pieces 483a, 483b which each have a shape approximating one quarter (or slightly more than one quarter) of a circle, with the two patch pieces 483a, 483b being positioned diametrically opposing each other aligned in about the same axial plane perpendicular to longitudinal axis X, at or near the leading end 489. In the embodiment shown in FIG. 35I, additional patch pieces are positioned within anchor 410, but not visible in FIG. 35I because the portion of the anchor 410 carrying the additional patch pieces is in a collapsed condition. For instance, FIGS. 35L and 35M illustrate third and fourth patch pieces 483c, 483d at a different axial alignment than first and second patch pieces 483a, 483b. In particular, FIG. 35L illustrates anchor 410 when fully expanded so that the trailing end 490 is relatively close to the leading end 489 compared to FIG. 35I. The third and fourth patch pieces 483c, 483d may each have a shape approximating one quarter (or slightly more than one quarter) of a circle, with the two patch pieces 483c, 483d being positioned diametrically opposing each other aligned in about the same axial plane perpendicular to longitudinal axis X, at or near the trailing end 489. The third and fourth path pieces 483c, 483d may be positioned circumferentially between (although slightly longitudinally offset from) the first and second patch pieces 483a, 483b. With this configuration, when the anchor 410 is in the fully expanded condition shown in FIG. 35L, a projection of the four patch pieces 483a-483d will create a complete (or nearly complete) circle or other continuous area of coverage of the area of the anchor 410. Such examples may allow for complete or substantially complete coverage of the area of the anchor to cover, or otherwise be positioned adjacent to, the apical puncture to help ensure bleeding is minimized, while maximizing sealing of the puncture, while simultaneously minimizing the bulk of the patch within the anchor to minimize the profile of the anchor in the collapsed condition. In other words, if the patch was formed as a single, substantially circular member, it would provide complete coverage of the area of the anchor 410, but, when the anchor is collapsed, there would be a relatively high volume of patch material in a small section of the collapsed anchor. By splitting the patch into multiple pieces along the length of the anchor, the volume is more evenly distributed when the anchor 410 is collapsed, reducing overall bulk created by the patch pieces, without sacrificing the area which the patch covers when the anchor is expanded.

FIGS. 35J and 35K illustrate anchor 410 in collapsed conditions, showing the two pairs of patch pieces are able to be collapsed at their respective axial locations without bunching with each other. In other words, when the anchor 410 is collapsed, first and second patch pieces, 483a, 483b positioned near leading end 489 may not bunch with third and fourth patch pieces 483c, 483d positioned at trailing end 490, thereby allowing the anchor to reach a smaller radial profile. The patch pieces are shown in FIG. 35K with first and second patch pieces 483a, 483b disposed at the leading end 489 and third and fourth patch pieces 483c, 483d at the trailing end 490 of anchor 410, however it is contemplated that the patch pieces may be positioned at any longitudinal location along the anchor, e.g., the longitudinal middle portion or anywhere in between the middle portion and the leading or trailing end. And although FIGS. 35I-L illustrate a patch that is formed as four separate substantially quarter-circle shaped pieces provided in two pairs at longitudinally spaced distances along the anchor 410, more or fewer pieces may be provided in two or more spaced axial distances.

It is contemplated that each patch piece may have any size and may be disposed at any longitudinal position within the anchor, the patch pieces still configured to cover an entire cross-sectional area of the anchor when projected onto a single plane as described above. For example, each patch piece may be sized to cover about one-fifth of the cross-sectional area. In such examples, at least some or all of the patch pieces may be positioned at different longitudinal locations (i.e., positioned a spaced distance apart in the longitudinal direction), to spread the bulk of the material out along the length of the anchor when in the collapsed condition. When the anchor is in the expanded condition, each patch piece is configured to cover a respective portion of the anchor's cross-sectional area such that the combination of five patch pieces may cover the entire cross-sectional area (at varying longitudinal locations). In further examples, each patch piece may be sized to cover about one-sixth of the cross-sectional area, with at least six patch pieces, some or all of which positioned at different longitudinal locations. It is further contemplated that the anchor may include more patch pieces than needed to cover the full cross-sectional area, such that one or more patch pieces overlap or are stacked on top of each other at different longitudinal positions. Such examples may provide layers of patch pieces in the longitudinal direction which may improve the coverage of the puncture by further reducing or preventing fluid from passing through the anchor while still minimizing the bunching of material in the collapsed condition.

In other examples, the patch pieces may extend along a plane oblique to longitudinal axis X. That is, each patch piece may be oriented such that it extends radially to cover a portion of the anchor's cross-sectional area and also extends axially (i.e., longitudinally). In other words, a first side, edge or corner of first patch piece 483a may be positioned within the anchor at a first axial cross-section, while a second side, edge or corner (e.g., opposite the first) of the first patch piece 483a may be positioned at a second axial cross-section proximal or distal to the first. For example, patch pieces in such an embodiment may form a helix or a spiral staircase along the anchor, such as the embodiments shown in FIGS. 35O and 35P. If the patch has this helical or spiral configuration, it may be provided as a single continuous piece and still achieve benefits of reducing the bulk of the anchor 410 at any one portion when collapsed, because the patch extends longitudinally. FIGS. 35O-P illustrate an embodiment of anchor 410 having first patch piece 483a and second patch piece 483b spiraling longitudinally along the anchor 410. First patch piece 483a may cover approximately half of the cross-sectional area when projected onto a plane and second patch piece 183b may cover the other half of the cross-sectional area not covered by first patch piece 483a in the expanded condition. It should be understood that a spiral or helically extending patch may also be provided that is formed of one or more than two pieces, as in the examples described above. For example, the anchor may have a first patch piece, a second patch piece, a third patch piece and a fourth patch piece, each patch piece spiraling longitudinally along the anchor and covering approximately a quarter of the cross-sectional area when projected onto a plane in the expanded condition. In any of the described embodiments, the patch pieces may overlap in the longitudinal direction such that a plurality of layers covers the puncture in at least some portions of the anchor. As noted above, overlapping layers may improve the coverage of the puncture to provide additional protection from fluids passing therethrough. It should be understood that the amount of fabric, number of patch pieces, and the angle at which the pieces are attached may be modified as needed to accommodate the shape and size of the anchor. The patch pieces may be formed of PET, nylon, polyurethane, PLLC, or the like. The chosen fabric may be modified as desired to improve tissue ingrowth.

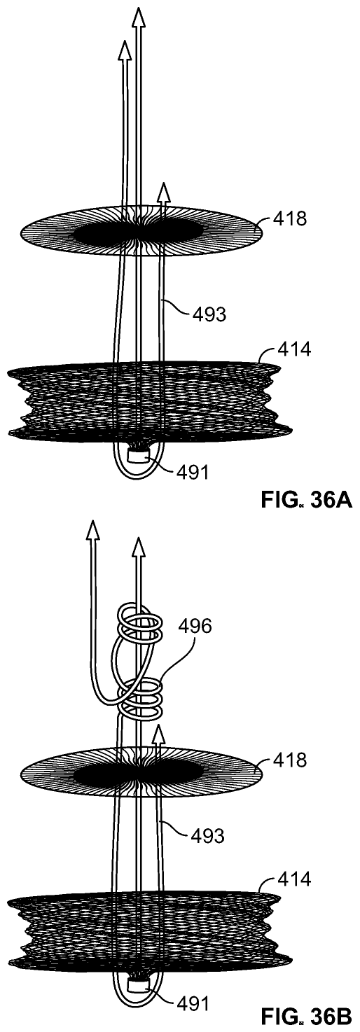

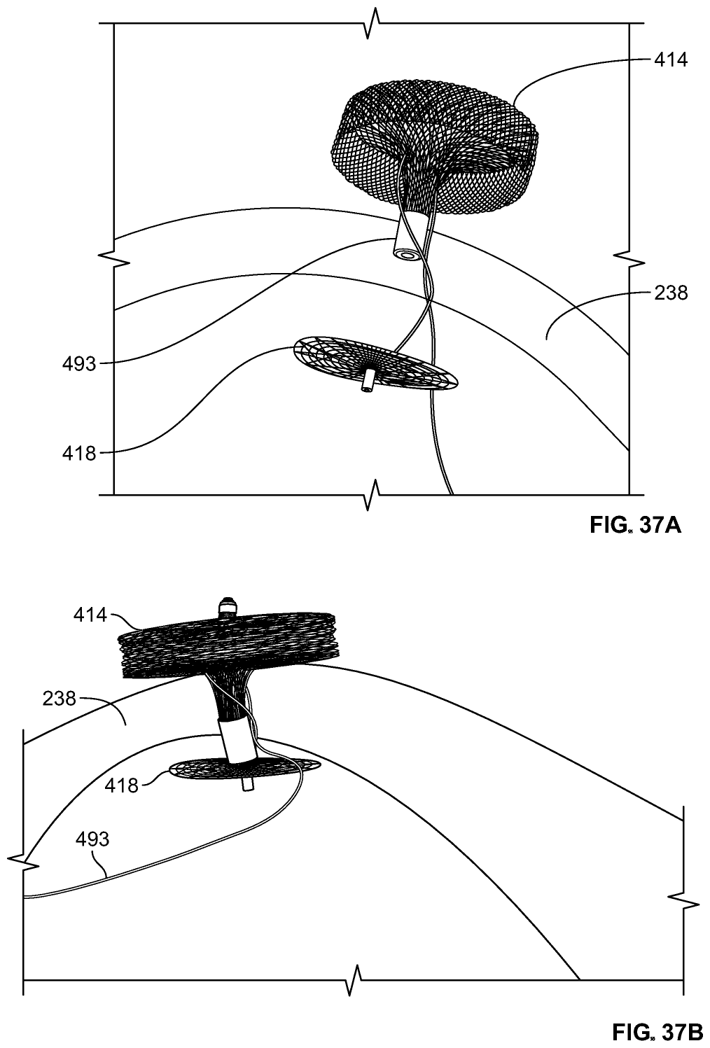

As noted above, cinch tether 493 may be operated by a user to secure locking disc 418 to the inside of the heart wall to sandwich the heart wall between first disc 414 and locking disc 418 to further seal the heart at the puncture and thereby minimize leakage and to further stabilize the position of the prosthetic heart valve. Cinch tether 493 may extend from locking disc 418 toward first disc 414 (i.e., the distal direction), passing through the ventricular wall and through first disc 414 to loop around or couple to leading hub 491 of first disc 414. After looping around or coupling to leading hub 491, cinch tether 493 may extend back in the first (i.e., proximal) direction passing a second time through first disc 414 and the ventricular wall and passing through locking disc 418 to extend proximally through the delivery device to be accessible to a user. After the first disc 414 is deployed on (or adjacent to) the outer wall of the ventricle, and the locking disc 418 is disposed on (or adjacent to) the inner wall of the ventricle, the user may pull on the proximal end of cinch tether 493 to apply a tension to cinch tether 493. Cinch tether 493 may be positioned adjacent to or internally within tether 426. In some examples, cinch tether 493 may be tied to tether 426 at a location along tether 426 proximal to locking disc 418 forming a slipknot 496 as shown in FIG. 36B. Slipknot 496 may permit cinch tether 493 to be tensioned by the user and prevent retraction of cinch tether 493 after tension is released. Tension may apply a simultaneous force to both first disc 414 and locking disc 418 toward each other, further securing anchor 410 to each side of the ventricular wall 238, as shown in FIGS. 37A-B. It is also contemplated that rather than compressing first disc 414 and locking disc 418 against ventricular wall 238 with cinch tether 493, first disc 414 may be coupled to locking disc 418 by a rigid structure. In such examples, the first disc 414 may be appropriately spaced from locking disc 418 and attached by the rigid structure such that the first and locking discs are spaced a fixed distance apart and may be in position to surround the puncture on each side of ventricular wall 238 when the anchor is deployed from the delivery device. In further examples, first disc 414 may be coupled to locking disc 418 by a pivoting ball structure such that the first and locking disc are a fixed distance apart and appropriately spaced upon delivery, but may still be able to adjust their relative angles to more accurately contour the ventricular walls 238.

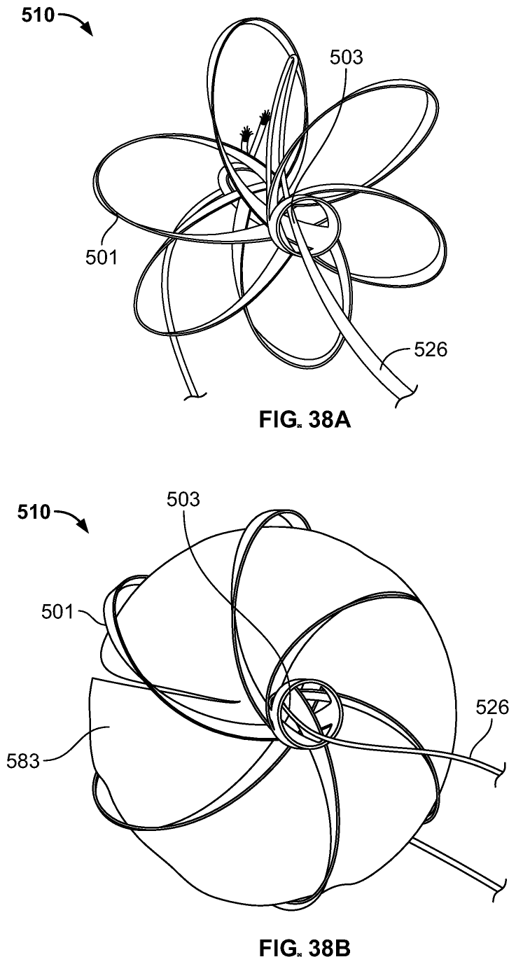

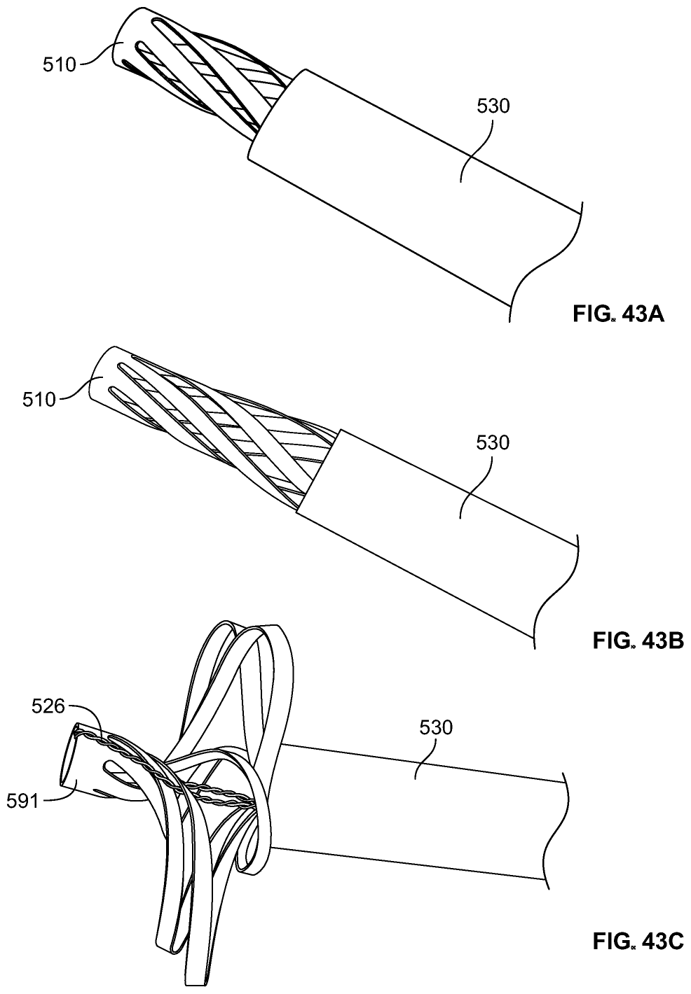

FIGS. 38A-B illustrate an anchor 510 according to another embodiment of the disclosure. Anchor 510 is configured to be delivered to the apex of the heart in a manner similar to the above-described delivery of anchors 210, 310 and 410. That is, anchor 510 may be compressed to be disposed within a delivery tube (described below with reference to FIGS. 43A-C) to be delivered transseptally, e.g., through trans-jugular or trans-femoral insertions as described above with reference to FIGS. 14-15. Anchor 510 may have a compressed delivery configuration while disposed within delivery tube 530, a resting expanded configuration when released from delivery tube 530, and a tensioned configuration when tether 526 is tensioned.

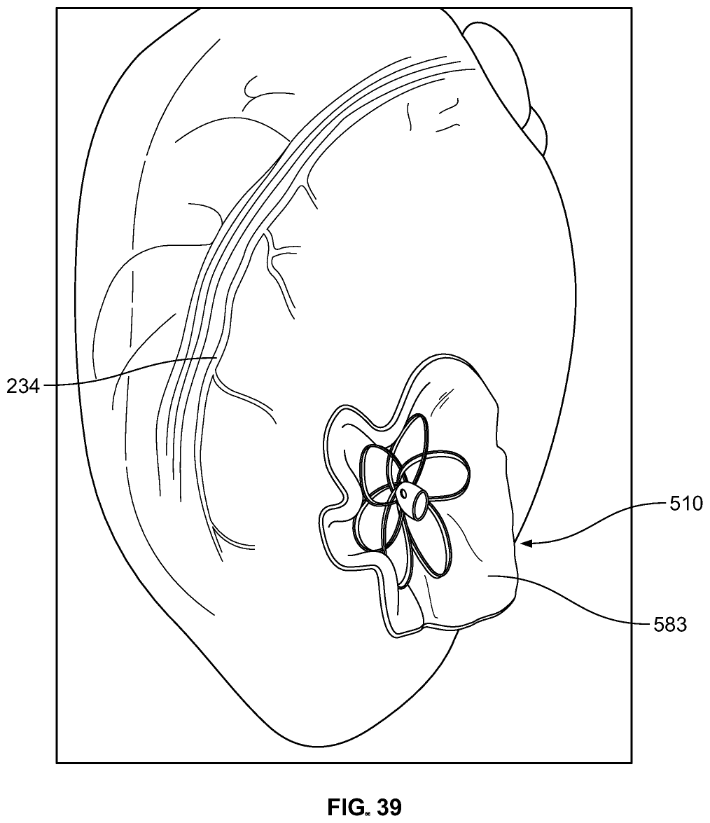

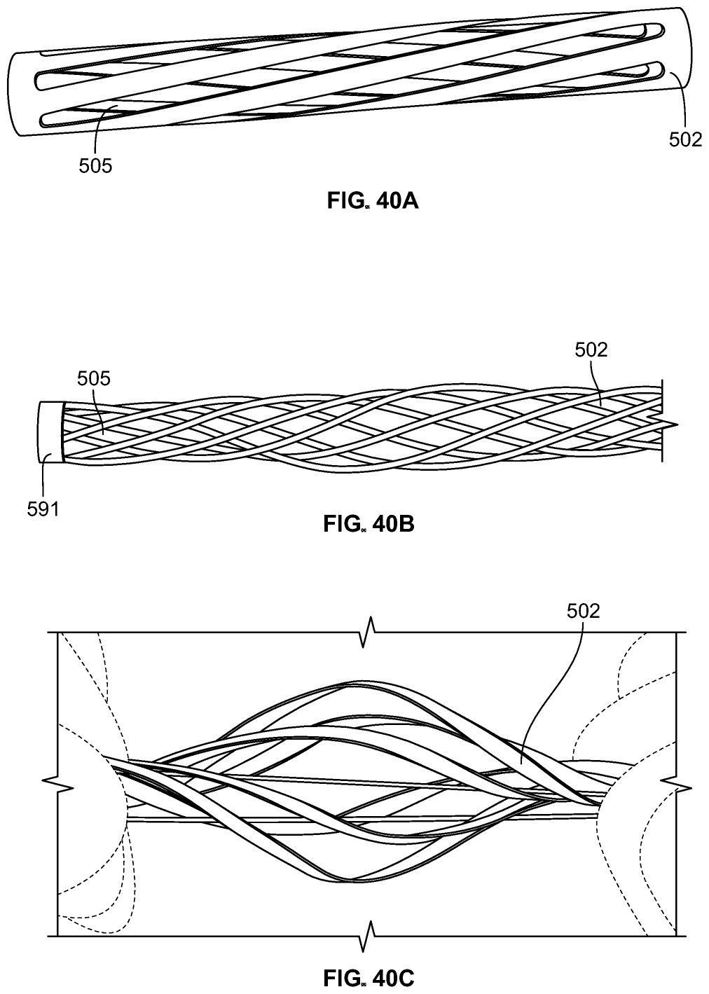

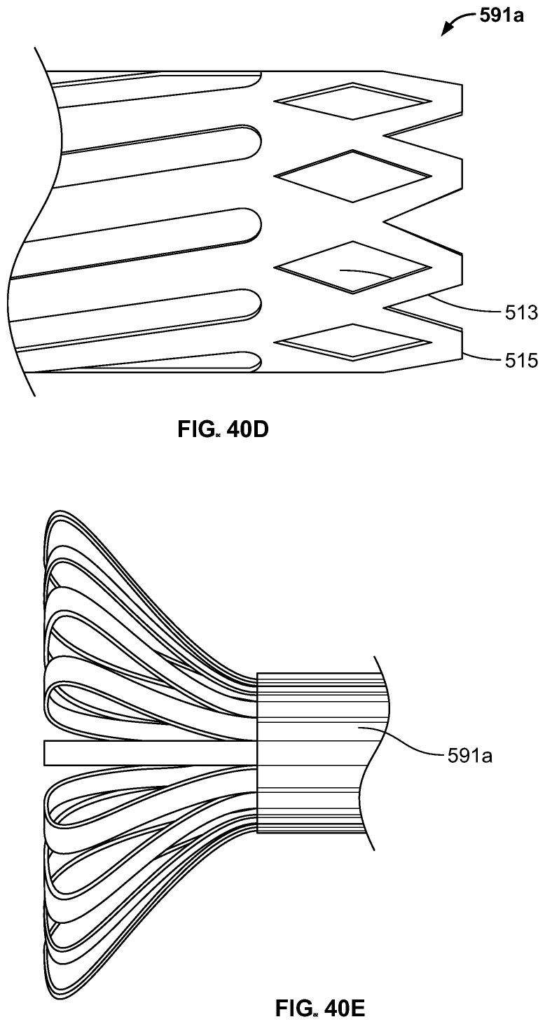

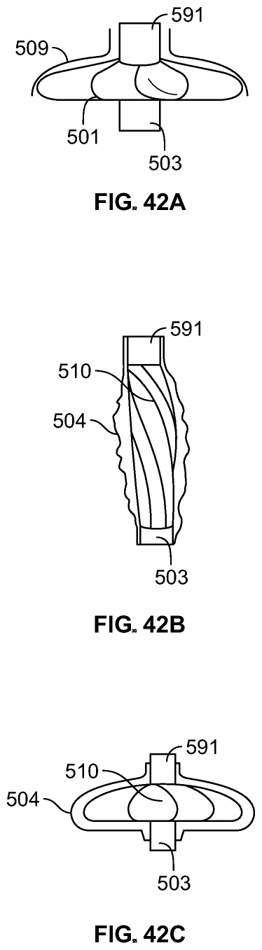

Anchor 510 may be formed of the same or similar material and means as described above with reference to anchor 310. For example, anchor 510 may be formed from a shape memory material (e.g., nitinol) which may be shaped (e.g., heat set) into a flower-like shape forming arms or petals 501 centered on axis X. Petals 501 may, for example, be cut (e.g. laser cut) from a nitinol tube 502, as shown in FIG. 40A (described below in greater detail), then heat set into a contoured profile as shown in FIG. 38A. In some examples, tube 502 from which anchor 510 is cut may measure between about 0.03 inches and about 0.20 inches in diameter and tube 502 may measure between about 0.005 inches and about 0.025 inches in wall thickness. In the illustrated embodiment, anchor 510 includes six petals 501. In other examples, anchor 510 may include between four and thirty-two petals. Anchor 510 may include a trailing hub 503 and a leading hub 591 (shown more clearly in FIG. 42A-C). In some examples, a hub may be formed as a hollow cylindrical structure having a continuous surface as shown, for example, by hub 591 in FIG. 40B. In other words, the hubs may be a part of the tube from which anchor 510 is cut, with the unprocessed ends of the tube forming the hubs. In other examples, the hubs may be further processed. In one example, the tube that forms the hubs at the ends of the tube may be cut (e.g., laser cut) or otherwise formed to have struts 515 defining cells 513 therebetween as shown, for example, by hub 591a in FIG. 40D. Such structure may enable the hub 591a to compress and expand. Hub 591a is shown in FIG. 40D after being cut (e.g. laser cut). After being formed as shown in FIG. 40D, the hub 591a may be shape-set to a smaller diameter, for example by heat setting into a compressed condition that has a smaller diameter than the original diameter of the unprocessed tube. For example, FIG. 40E illustrates the hub 591a after being heat-set into a smaller diameter configuration, with the original diameter of the unprocessed tube being illustrated by the dotted line in FIG. 40E. Compression of the hub may reduce the overall radial profile of the anchor when the anchor is in the compressed delivery configuration, minimizing the diameter of a delivery tube in which the anchor is disposed. Tether 526 may extend and pass through trailing hub 503 to be sutured or otherwise coupled to leading hub 591. Similar to anchor 410, tension from tether 526 may be applied to anchor 510 directly at leading hub 591, without direct application of tension to trailing hub 503, to distribute the force of tension throughout anchor 510 and around the puncture in the ventricular wall, thereby minimizing the force applied directly to the puncture.

Anchor 510 may further include a fabric or other sealing member, such as patch 583 as shown in FIGS. 38B and 39. Patch 583 may have a leading face which may face away from the heart when anchor 510 is in the implanted configuration, and a trailing face which may face toward the heart when anchor 510 is in the implanted configuration. Patch 583 may be disposed internally within petals 501, as shown in FIG. 38B, such that each petal 501 has a first portion extending along (and/or adjacent) a trailing face of patch 583, loops over the perimeter of patch 583 and further has a second portion extending along (and/or adjacent) leading face of patch 583. In other examples, patch 583 may be disposed external to anchor 510 between anchor 510 and the heart in an implanted configuration at shown in FIG. 39. In either case, it may be desirable to couple the patch 583 to the anchor 510, for example by suturing the patch 583 to portions of the anchor 510 that form the petals 501.

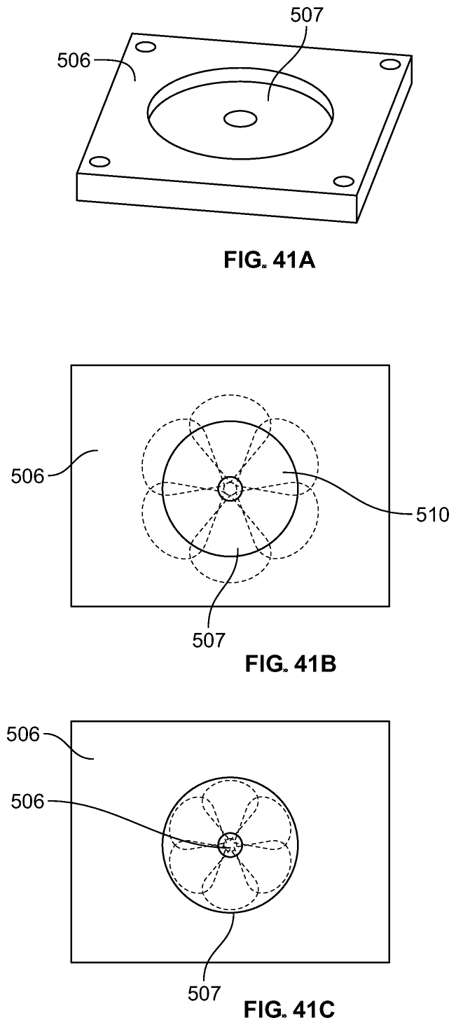

Petals 501 are formed from tube 502 shown in FIG. 40A, which may be substantially straight having spiral cuts 505 thereon. Each spiral cut 505 may extend rotationally approximately 360 degrees around tube 502 as spiral cuts 505 extend between first and second ends of tube 502. It should be understood that the length of spirals 505, as well as the degree of axial compression of the tube 502, may determine the diameter of petals 501. Tube 502 may be axially compressed to form anchor 510 having petals 501. The petals begin to form upon compression of tube 502, as shown in FIGS. 40B-C which illustrate a partially compressed tube. While compressing tube 502, petals 501 may be formed with unequal dimensions or undesired sizes. Thus, in some embodiments, petals 501 may be placed in a block or mold 506 defining a circular recess 507 as shown in FIGS. 41A-C. Petals 501 may expand beyond the diameter of circular recess 507 as shown in FIG. 41B, and may be positioned within circular recess 507 to form substantially similar dimensions (e.g., width, radii) among each petal 501 as shown in FIG. 41C. For example, FIG. 41B illustrates that the radial tip of each petal may be relatively sharp or pointed prior to placement within the mold 506, but upon placement within the mold 506, the radial tips of the petal become more blunted (e.g. a larger radius of curvature at the tip of the petal). Petals 510 may be heat set in the shape formed by block 506 to maintain the set shape after being removed from block 506. In some embodiments, the struts forming the petals 501 may have certain desired relative dimensions. For example, the cut width of the strut forming each petal 501 is preferably greater than the thickness of the strut forming each petal. In this context, the cut width refers to the circumferential edge-to-edge distance remaining for each strut when the tube of material is cut to form each strut. In other words, the cut width of the strut is the distance of the radially outer or inner surface of the strut, the distance being measured transverse the axial length of the strut. The thickness of the strut, on the other hand, in this context refers to the wall thickness of the tube from which the struts are cut. In other words, the thickness of the struts forming the petals 501 may be the distance between the radially-inward and radially-outward facing surfaces of the particular strut. The natural twist of petals 501 may aid in the collapsibility of anchor 510 around hubs 503, 591.

In some examples, fabric 509 may be added to the exterior of tube 502 in a disc form (or any other suitable shape) covering approximately half the radial length of petals 501 such that, for example, the fabric 509 covers a leading face of the petals, as shown in FIG. 42A. In further examples, anchor 510 may include a stretchable bag 504 positioned over the exterior of the tube before the tube is compressed, as shown in FIG. 42B. The tube may be compressed within bag 504 such that petals 501 are formed covered by the bag as shown in FIG. 42C. Bag 504 may be made of a material suitable for stretching, such as urethane, tecothane, santoprene or the like. It should be understood that the term “bag” need not require any specific structure, but may take any suitable form that is capable of covering the anchor 510 when it is compressed and also expanded.

Anchor 510 may be deployed from delivery tube 530 in a manner substantially similar to that of anchors 210, 310 and 410 as described above. In FIG. 43A, anchor 510 is shown disposed substantially inside delivery tube 530. Anchor 510 may be deployed from delivery tube 530 by a push rod (e.g., push rod 466 shown in FIG. 33A). Upon being delivered to the left ventricle of the heart, delivery tube 530 (or another component of the delivery system) may puncture the ventricular wall and pass through to deploy anchor 510 outside of the heart. In some examples, leading hub 591 may be sufficiently sharp in the collapsed configuration to puncture the hole in the ventricular wall. The distal end of delivery tube 530 may be advanced beyond the heart into the intercostal space and anchor 510 may be partially deployed from delivery tube 530 as shown in FIG. 44B by translating the push rod toward the leading (i.e., distal) end of tube 530 and contacting anchor 510. As anchor 510 is deployed from delivery tube 530, a tension may be applied to tether 526 as shown in FIG. 43C. The tension on tether 526 may retract or hold substantially in place the leading end of anchor 510 as the push rod continues to deploy anchor 510 from delivery tube 530. FIG. 43C illustrates anchor 510 substantially deployed from tube 530 with continued tension applied to tether 526, beginning to form petals 501. Such a deployment method may minimize the distance beyond the wall of the heart into the intercostal space through which anchor 510 extends during deployment, similar to the above description of the delivery of anchor 410.

Anchor 510 is shown in FIG. 39 fully deployed from delivery tube 530 in an implanted tethered configuration. It is contemplated that anchor 510 may include a locking disc and a cinch tether substantially similar to second disc 418 and cinch tether 493 described above.

According to an aspect of the disclosure, a prosthetic heart valve system comprises:

a prosthetic heart valve;

a tether having a first end and a second end, the prosthetic heart valve configured to receive the first end of the tether; and

a collapsible and expandable anchor comprising:
a center rim configured to couple to the second end of the tether;
a plurality of struts extending radially outward from the center rim forming an inner dome that is concave toward a heart when the anchor is in a resting configuration implanted on an apex of the heart; and
a plurality of wings extending radially outward from the struts; and/or

the wings define eyelets formed on a radially outward end of the wings, each eyelet defining a radially terminal end of the anchor; and/or

the wings extend in a direction away from the heart when the anchor is in the resting configuration implanted on the apex of the heart; and/or

the anchor includes a fabric and the tether is coupled to the fabric through an aperture at the center rim of the anchor; and/or

the wings extend substantially orthogonal to a central longitudinal axis passing through the center rim of the anchor; and/or

the tether is configured to transition the anchor from the resting configuration to a tethered configuration upon application of tension to the tether while the anchor is on the apex of the heart; and/or

the inner dome is has a reduced concavity when the anchor is in the tethered configuration relative to the resting configuration; and/or

the inner dome is substantially flat when the anchor is in the tethered configuration; and/or

the wings extend substantially orthogonal to a central longitudinal axis passing through the center rim of the anchor in the tethered configuration; and/or

when the anchor is received within a delivery device in a collapsed delivery configuration, the center rim is at a leading distal end of the anchor and the wings are at a proximal trailing end of the anchor; and/or

when the anchor is received within a delivery device in a collapsed delivery configuration, the center rim is at a trailing proximal end of the anchor and the wings are at a leading distal end of the anchor.

According to another aspect of the disclosure, a prosthetic heart valve system comprises:

a prosthetic heart valve;

a tether having a first end configured to connect to the prosthetic heart valve and a second end; and

a collapsible and expandable anchor comprising:
a leading face; and
a trailing face;
wherein the second end of the tether is configured to couple directly to the leading face and does not directly couple to the trailing face of the body, the tether configured to transition the anchor from a resting configuration to a tethered configuration by tensioning the tether to apply a tensioning force directly to the leading face; and/or

the anchor includes a leading hub at the leading face and a trailing hub at the trailing face, the tether configured to pass through the trailing hub and to couple directly to the leading hub; and/or

the anchor is formed of braided strands of wire, and in the resting configuration of the anchor, the anchor forms a disc having a radially inner portion, a middle portion positioned radially outward of the radially inner portion, and a rim positioned radially outward of the middle portion, the middle portion forming bridge portions and pockets, the bridge portions having higher concentrations of the strands of wire than the pockets; and/or

the strands of wire are distributed substantially uniformly within the radially inner portions and within the rim; and/or

a fabric patch positioned internally within the anchor and a fabric cuff positioned external to the anchor adjacent the trailing face of the anchor; and/or

the anchor includes a locking disc positioned a spaced distance proximal to the trailing face, the locking disc fixed relative to the trailing face and configured to abut the interior of a ventricular wall in a deployed configuration; and/or

the anchor includes a locking disc coupled to a first end of a cinch tether, the cinch tether extending from the first end distally toward the leading face, the cinch tether looping around the leading hub and extending back proximally toward the locking disc and to a second end of the cinch tether configured to be accessible to a user; and/or

the locking disc is configured to be positioned inside a heart against an interior of a ventricular wall of the heart, and the cinch tether is configured to be tensioned to apply a force to draw the anchor and the locking disc toward each other to sandwich the ventricular wall therebetween; and/or

in the resting configuration of the anchor, the anchor includes a plurality of arms extending radially outward from a radial center of the anchor, the anchor further including an initial configuration in which the arms are formed from a cylindrical tube having spiral cuts thereon, each spiral cut extending longitudinally along the tube and rotationally approximately 360 degrees around the tube, the tube forming petal-shaped arms when the anchor is in the resting configuration; and/or

a plurality of fabric patch pieces positioned internally within the anchor, the patch pieces sized and shaped to cover a full cross-sectional area of the anchor when the anchor is in an expanded condition and the patch pieces are projected onto a single plane perpendicular to a longitudinal axis; and/or

in the expanded condition, each patch piece lies in a plane extending perpendicular to a longitudinal axis of the anchor, and at least one patch piece is positioned a spaced distance from another patch piece in a longitudinal direction; and/or

at least one patch piece lies in a plane extending at an angle oblique to a longitudinal axis of the anchor.

According to another aspect of the disclosure, a method of implanting an anchor on an external surface of a heart for securing a prosthetic heart valve within the heart comprises:

collapsing the anchor into a collapsed delivery configuration within a delivery tube, the anchor having a leading face and a trailing face, a tether being attached to the leading face;

advancing the delivery tube into a ventricle of the heart;

after advancing the delivery tube into the ventricle, passing a distal end of the delivery tube through a ventricular wall of the heart; and

after passing the distal end of the delivery tube through the ventricular wall, deploying the anchor from the distal end of the delivery tube onto an apex of the heart by tensioning the tether to restrain movement of the leading face relative to the ventricular wall deploying remaining portions of the anchor to limit a distance which the anchor protrudes beyond the ventricular wall during deployment; and/or

collapsing a locking disc of the anchor into a collapsed delivery configuration within the delivery tube so that the locking disc is positioned proximal to the trailing face of the anchor within the delivery tube; and/or

after the anchor is deployed, retracting the distal end of the delivery tube back into the ventricle and deploying the locking disc from the distal end of the delivery tube into the ventricle; and/or

after deploying the locking disc, tensioning a cinch tether to apply a force to draw the trailing face and the locking disc toward each other to sandwich the ventricular wall between the trailing face and the locking disc.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve system, comprising:
a prosthetic heart valve;
a tether having a first end and a second end, the prosthetic heart valve configured to receive the first end of the tether; and
a collapsible and expandable anchor comprising:
a center rim configured to couple to the second end of the tether;
a plurality of struts extending radially outward from the center rim forming an inner dome that is concave toward a heart when the anchor is in a resting configuration implanted on an apex of the heart; and
a plurality of wings extending radially outward from the struts.

2. The prosthetic heart valve system of claim 1, wherein the wings define eyelets formed on a radially outward end of the wings, each eyelet defining a radially terminal end of the anchor.

3. The prosthetic heart valve system of claim 1, wherein the wings extend in a direction away from the heart when the anchor is in the resting configuration implanted on the apex of the heart.

4. The prosthetic heart valve system of claim 1, wherein the anchor includes a fabric and the tether is coupled to the fabric through an aperture at the center rim of the anchor.

5. The prosthetic heart valve system of claim 1, wherein the wings extend substantially orthogonal to a central longitudinal axis passing through the center rim of the anchor.

6. The prosthetic heart valve system of claim 1, wherein the tether is configured to transition the anchor from the resting configuration to a tethered configuration upon application of tension to the tether while the anchor is on the apex of the heart.

7. The prosthetic heart valve system of claim 6, wherein the inner dome has a reduced concavity when the anchor is in the tethered configuration relative to the resting configuration.

8. A prosthetic heart valve system, comprising:
a prosthetic heart valve;
a tether having a first end configured to connect to the prosthetic heart valve and a second end; and
a collapsible and expandable anchor comprising:
a leading face; and
a trailing face;
wherein the second end of the tether is configured to couple directly to the leading face and does not directly couple to the trailing face of the body, the tether configured to transition the anchor from a resting configuration to a tethered configuration by tensioning the tether to apply a tensioning force directly to the leading face.

9. The prosthetic heart valve system of claim 8, wherein the anchor includes a leading hub at the leading face and a trailing hub at the trailing face, the tether configured to pass through the trailing hub and to couple directly to the leading hub.

10. The prosthetic heart valve system of claim 8, wherein the anchor is formed of braided strands of wire, and in the resting configuration of the anchor, the anchor forms a disc having a radially inner portion, a middle portion positioned radially outward of the radially inner portion, and a rim positioned radially outward of the middle portion, the middle portion forming bridge portions and pockets, the bridge portions having higher concentrations of the strands of wire than the pockets.

11. The prosthetic heart valve system of claim 8, further comprising a fabric patch positioned internally within the anchor and a fabric cuff positioned external to the anchor adjacent the trailing face of the anchor.

12. The prosthetic heart valve system of claim 8, wherein the anchor includes a locking disc positioned a spaced distance proximal to the trailing face, the locking disc fixed relative to the trailing face and configured to abut the interior of a ventricular wall in a deployed configuration.

13. The prosthetic heart valve system of claim 8, wherein the anchor includes a locking disc coupled to a first end of a cinch tether, the cinch tether extending from the first end distally toward the leading face, the cinch tether looping around the leading hub and extending back proximally toward the locking disc and to a second end of the cinch tether configured to be accessible to a user.

14. The prosthetic heart valve system of claim 13, wherein the locking disc is configured to be positioned inside a heart against an interior of a ventricular wall of the heart, and the cinch tether is configured to be tensioned to apply a force to draw the anchor and the locking disc toward each other to sandwich the ventricular wall therebetween.

15. The prosthetic heart valve system of claim 8, wherein in the resting configuration of the anchor, the anchor includes a plurality of arms extending radially outward from a radial center of the anchor, the anchor further including an initial configuration in which the arms are formed from a cylindrical tube having spiral cuts thereon, each spiral cut extending longitudinally along the tube and rotationally approximately 360 degrees around the tube, the tube forming petal-shaped arms when the anchor is in the resting configuration.

16. The prosthetic heart valve system of claim 8, further comprising a plurality of fabric patch pieces positioned internally within the anchor, the patch pieces sized and shaped to cover a full cross-sectional area of the anchor when the anchor is in an expanded condition and the patch pieces are projected onto a single plane perpendicular to a longitudinal axis.

17. The prosthetic heart valve system of claim 16, wherein in the expanded condition, each patch piece lies in a plane extending perpendicular to a longitudinal axis of the anchor, and at least one patch piece is positioned a spaced distance from another patch piece in a longitudinal direction.

18. A method of implanting an anchor on an external surface of a heart for securing a prosthetic heart valve within the heart, comprising:
collapsing the anchor into a collapsed delivery configuration within a delivery tube, the anchor having a leading face and a trailing face, a tether being attached to the leading face;
advancing the delivery tube into a ventricle of the heart;
after advancing the delivery tube into the ventricle, passing a distal end of the delivery tube through a ventricular wall of the heart; and
after passing the distal end of the delivery tube through the ventricular wall, deploying the anchor from the distal end of the delivery tube onto an apex of the heart by tensioning the tether to restrain movement of the leading face relative to the ventricular wall deploying remaining portions of the anchor to limit a distance which the anchor protrudes beyond the ventricular wall during deployment.

19. The method of claim 18, further comprising
collapsing a locking disc of the anchor into a collapsed delivery configuration within the delivery tube so that the locking disc is positioned proximal to the trailing face of the anchor within the delivery tube; and
after the anchor is deployed, retracting the distal end of the delivery tube back into the ventricle and deploying the locking disc from the distal end of the delivery tube into the ventricle.

20. The method of claim 19, further comprising
after deploying the locking disc, tensioning a cinch tether to apply a force to draw the trailing face and the locking disc toward each other to sandwich the ventricular wall between the trailing face and the locking disc.
